(19) United States
(12) Patent Application Publication
(10) Pub. No.: US 2013/0059773 A1
(43) Pub. Date: Mar. 7, 2013

(54) COMPOUNDS FOR THE TREATMENT OF INFLAMMATION AND NEUTROPENIA

(71) Applicant: Veronique Witko-Sarsat, Paris (FR)

(72) Inventor: Veronique Witko-Sarsat, Paris (FR)

(21) Appl. No.: 13/580,756

(22) PCT Filed: Feb. 24, 2011
(86) PCT No.: PCT/EP2011/052760

§ 371 Date: Nov. 8, 2012

(30) Foreign Application Priority Data
Feb. 24, 2010 (EP) 10305182.7

(51) Int. Cl. A61K 38/16; G01N 33/566; A61P 31/04; A61P 17/02; A61P 1/04; A61P 9/10; A61P 19/06; A61P 19/02; A61K 38/10; A61K 38/08; C07K 7/06; C07K 7/08; C07K 14/00; A61P 11/00; A61P 1/00; A61P 41/00; G01N 33/53

(52) U.S. Cl. 514/2.4; 435/7.92; 436/501; 514/9.4; 514/13.2; 514/16.4; 514/16.7; 514/16.8; 514/21.3; 514/21.4; 514/21.5; 514/21.6; 514/21.7; 514/21.8; 530/324; 530/325; 530/326; 530/327; 530/328; 530/329

(57) ABSTRACT

The present invention concerns compounds modulating apoptosis of neutrophil cells. In particular, the invention concerns compounds inhibiting an interaction between Proliferating Cell Nuclear Antigen (PCNA) and proteins binding to cytoplasmic PCNA in neutrophil cells, for use in the treatment of a disease involving a neutrophil-dependent inflammatory process. The invention also relates to a method for the identification of a compound for use in the treatment of a neutrophil-dependent inflammatory process. The invention further relates to peptides for use in the treatment of neutropenia.

The present invention concerns compounds modulating apoptosis of neutrophil cells. In particular, the invention concerns compounds inhibiting an interaction between Proliferating Cell Nuclear Antigen (PCNA) and proteins binding to cytoplasmic PCNA in neutrophil cells, for use in the treatment of a disease involving an inflammatory process. The invention also relates to a method for the identification of a compound for use in the treatment of a neutrophil-dependent inflammatory process. The invention further relates to peptides for use in the treatment of neutropenia.

### BACKGROUND OF THE INVENTION

### The Proliferating Cell Nuclear Antigen (PCNA)

Proliferating cell nuclear antigen (PCNA) is a crucial factor in DNA synthesis and repair, initially characterized as the auxiliary protein of DNA polymerases delta and epsilon. All the PCNA functions described until now exclusively reflect its nuclear localization. Within the last few years, many proteins have been found to interact with PCNA, including various enzymes and regulatory proteins like CDKs or the CDK-inhibitor p21/waf1 (Waga et al. 1994; Nature 369:574-578). As a corollary, it has been hypothesized that PCNA could play a role in cellular pathways other than replication, e.g. in nucleotide-excision repair, mismatch repair, cell cycle and apoptosis, thus acting as a “cellular communicator” connecting all these important cellular processes.

Neutrophils and Inflammation

Neutrophils are terminally differentiated effector cells, whose principal function is to migrate to sites of inflammation, where they exert anti-infectious and proinflammatory effects. However, a growing body of evidence shows that neutrophils can modulate the inflammatory process (Nathan et al. 2006; Nat. Rev. Immunol. 6:173-182).

Notably, they can both synthesize a wide variety of cytokines involved in immunoregulation and proteins increasing their lifespan, e.g. Mcl-1, A1/Bf1-1, annexin-1 and galectin, to perform efficiently their functions in host defense. However, once their effector role is terminated, their temporally regulated apoptosis, followed by phagocytosis by tissue macrophages, is necessary for successful inflammation resolution (Rossi et al. 2007; Biochem. Soc. Trans. 35:288-291). Thus, neutrophil apoptosis is a pivotal participant in inflammation resolution, even though its molecular mechanisms remain incompletely understood (Simon et al. 2003; Immunol. Rev. 193:101-110). Unlike macrophages or dendritic cells, neutrophils do not proliferate and have a short lifespan. Yet, they express several cell-cycle regulatory proteins, like cyclin-dependent kinase-2 (CDK2), p27 and survivin, which, in view of the close relationships between proliferation and apoptosis, neutrophils might use to regulate their own survival.

Neutrophils do not proliferate and survive for a very short time in the absence of any infection (about six hours). However, on sites on which an infection is taking place, the survival time of neutrophils is significantly increased. However, accumulation of neutrophils is detrimental to the infected organism. The neutrophils must thus be efficiently destroyed by macrophages (by phagocytosis). This step of phagocytosis is an essential step for resolving inflammation. If it does not take place in a proper manner, the inflammation is not properly resolved, and it may lead to a chronic inflammation.

It is thus desirable to obtain compounds capable of promoting apoptosis of neutrophils, such compounds allowing the treatment of inflammation.

Neutrophils and Neutropenia

Neutropenia is a reduction in the blood neutrophil (granulocyte) count. Neutropenia can be caused by intrinsic defects in myeloid cells or their precursors. Neutropenia can also results from use of certain drugs, bone marrow infiltration or replacement, certain infections, or immune reactions. The most common causes include the administration of drugs, infections and marrow infiltrative processes

If neutropenia is severe, the risk and severity of bacterial and fungal infections increase. When neutrophil counts fall to under 500 μL, endogenous microbial flora (eg, in the mouth or gut) can cause infections. If the count falls to under 200/μL, inflammatory response may be nonexistent. Acute, severe neutropenia, particularly if another factor (e.g. cancer) also impairs the immune system, predisposing to rapidly fatal infections.

It is thus desirable to obtain compounds capable of preventing apoptosis of neutrophils, such compounds allowing the treatment of neutropenia.

### DESCRIPTION OF THE INVENTION

The inventors have found that mature neutrophils express high levels of PCNA, which was surprisingly exclusively localized in neutrophil cytosol. In addition, PCNA was constitutively associated with procaspases, presumably to prevent their activation. Notably, cytosolic PCNA levels changed in parallel with neutrophil survival rate. Indeed, cytosolic PCNA levels decreased during apoptosis, and increased during in vitro or in vivo exposure to the survival factor G-CSF.

Remarkably, it was found that competing peptides derived from the cyclin-dependent kinase inhibitor p21 trigger neutrophil apoptosis, thus demonstrating that specific modulation of PCNA protein interactions affect neutrophil survival.

It was further found that peptides derived from PCNA could also be used to modulate PCNA protein interactions and can either induce or prevent neutrophil apoptosis.

PCNA was also expressed in murine bone-marrow neutrophils but only weakly in apoptosis-prone neutrophils, collected during LPS-elicited lung inflammation resolution. Moreover, PCNA overexpression rendered neutrophil-differentiated PLB985 myeloid cells significantly more resistant to TRAIL- or gliotoxin-induced apoptosis. A mutation in the PCNA interdomain connecting loop, binding site for many partners, significantly decreased the PCNA-mediated anti-apoptotic effect. These results identify PCNA as regulator of neutrophil lifespan, thereby highlighting a novel target to modulate pathological inflammation or neutropenia.

Thus, the inventors identified an unexpected anti-apoptotic role for PCNA in neutrophils which, interestingly, express PCNA exclusively in their cytosol. Compounds inhibiting or triggering the biological activity of PCNA could thus be used for treating inflammation or neutropenia, respectively.

Compounds for Use in the Treatment of a Disease Involving an Inflammatory Process

The invention therefore pertains to a compound inhibiting an interaction between Proliferating Cell Nuclear Antigen (PCNA) and at least one polypeptide liable to bind to PCNA, in particular liable to bind to cytoplasmic PCNA in neutrophil cells, for use in the treatment of a disease involving an inflammatory process.

The compound according to the invention may for example correspond to a peptide, a small molecule, an antibody or an aptamer.

In a preferred embodiment, the compound is a peptide. By “peptide” is meant a chain of amino acids (including natural, modified and/or unusual amino acids) being at most 50 amino acids long. The peptide can for example have a length of about 6 to 50, 10 to 50, or 15 to 50, 20 to 50, 6 to 30, 10 to 30, 15 to 30, 20 to 30, or 15 to 25 amino acids.

A peptide according to the invention can for example correspond to a fragment of at least 6, 10, 15 or 20 consecutive amino acids of PCNA or of a polypeptide liable to bind to PCNA such as e.g. p21, Rfc1, Rfc3, FEN-1, DNA ligase-1, topoisomerase II alpha, Cdt1, Rrm3, WRN, RECQ5, UNG2, MPG, hMYH, APE2, XPG, CAF-1, PARP-1, WSTF, DNMT1, Eco1, Chl1, p57, ING1b, or p53.

In a preferred embodiment, the peptide can comprise or consist of a fragment of at least 6, 10, 15 or 20 consecutive amino acids of PCNA. Such a fragment preferably comprises at least 6, 10, 15 or 20 consecutive amino of the interdomain connecting loop of PCNA. Indeed, as shown in Example 9, peptides 3 and 4, which comprise a PCNA fragment of SEQ ID NO: 3, are capable of triggering neutrophil apoptosis. Thus, the peptide may for example comprise or consist of a sequence SEQ ID NO: 3, or a sequence at least 80, 85, 90 or 95% identical thereto, or a fragment of at least 6, 10, 15 or 20 consecutive amino acids thereof.

Alternatively, the peptide can comprise or consist of a fragment of at least 6, 10, 15 or 20 consecutive amino acids of p21. Such a fragment preferably comprises at least 6, 10, 15 or 20 consecutive amino acids of the p21 fragment of SEQ ID NO: 23, which spans from residue 141 to 160 of p21. Indeed, as shown in Example 6, the carboxyp21 peptide, which comprises sequence of SEQ ID NO: 23, is capable of triggering neutrophil apoptosis. Thus, the peptide may for example comprise or consist of the sequence SEQ ID NO: 23, or a sequence at least 80, 85, 90 or 95% identical thereto, or a fragment of at least at least 6, 10, 15 or 20 consecutive amino acids thereof.

The peptide according to the invention may further comprise a tag, e.g. a tag enhancing entry of the peptide into cells. The tag may for example comprise or consist of a tag derived from the HIV-1 Tat polypeptide. Preferably, the tag comprises or consists of a tag of SEQ ID NO: 27 (preferably located at the N-terminal extremity of the peptide) or of a tag of SEQ ID NO: 8 (preferably located at the C-terminal extremity of the peptide). SEQ ID Nos. 11, 12 and 24 are specific examples of peptides according to the invention consisting of a fragment of at least 6 consecutive amino acids of PCNA or p21 fused to a tag.

The peptide may further comprise chemical modifications, such as e.g. acetylation, in order to enhance its solubility or biodisponibility.

Therefore, the invention pertains to a peptide, preferably a peptide comprising or consisting of:
a) an amino acid sequence consisting of SEQ ID NO: 3 or 23;
b) an amino acid sequence consisting of SEQ ID NO: 3 or 23 fused to a tag;
c) an amino acid sequence consisting of SEQ ID NO: 11, SEQ ID NO: 12 or SEQ ID NO: 24;
d) a sequence homologous to the sequence of any one of (a) to (c) and presenting at least 80% of identity with said sequence of any one of (a) to (c); or
e) an amino acid sequence consisting of a fragment of at least 6, 10, 15 or 20 consecutive amino acids of the sequence of any one of (a) to (c); for use in the treatment of a disease involving an inflammatory process.

When the peptide is derived from another protein than PCNA, the peptide according to the invention is preferably liable to bind to PCNA, in particular to cytoplasmic PCNA in neutrophil cells.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% (5 of 100) of the amino acid residues in the subject sequence may be inserted, deleted, or substituted with another amino acid.

Methods for comparing the identity and homology of two or more sequences are well known in the art. The <<needle>> program, which uses the Needleman-Wunsch global alignment algorithm (Needleman and Wunsch, 1970 J. Mol. Biol. 48:443-453) to find the optimum alignment (including gaps) of two sequences when considering their entire length, may for example be used. The needle program is for example available on the ebi.ac.uk world wide web site. The percentage of identity in accordance with the invention is preferably calculated using the EMBOSS::needle (global) program with a “Gap Open” parameter equal to 10.0, a “Gap Extend” parameter equal to 0.5, and a Blosum62 matrix.

Peptides comprising or consisting of an amino acid sequence “at least 80%, 85%, 90% or 95% identical” to a reference sequence may comprise mutations such as deletions, insertions and/or substitutions compared to the reference sequence. In a preferred embodiment, the mutation corresponds to a conservative substitution as indicated in the table below.

| Conservative | |
|---|---|
| substitutions | Type of Amino Acid |
| Ala, Val, Leu, Ile, | Amino acids with aliphatic hydrophobic side chains |
| Met, Pro, Phe, Trp | |
| Ser, Tyr, Asn, Gln, | Amino acids with uncharged but polar side chains |
| Cys | |
| Asp, Glu | Amino acids with acidic side chains |
| Lys, Arg, His | Amino acids with basic side chains |
| Gly | Neutral side chain |

Throughout the present specification, the term “PCNA” refers to the human Proliferating Cell Nuclear Antigen protein. In a preferred embodiment, “PCNA” refers to a protein of sequence SEQ ID NO: 1. However, this term also encompasses allelic variants and splice variants of the protein of SEQ ID NO: 1. In the frame of the present invention, the PCNA protein is preferably a cytoplasmic PCNA, most preferably a cytoplasmic PCNA found in neutrophil cells.

The expression “polypeptide liable to bind PCNA” refers to a protein that is capable of interacting with PCNA, preferably with cytoplasmic PCNA in neutrophil cells. Studies aiming at screening for peptides having a strong affinity for the interdomain connecting loop have been performed using peptide banks (Warbrick 2006, Oncogene 25:2850-2859). p21 is among those that bind with the highest affinity to PCNA (Moldovan et al. 2007; Cell 129:665-679). Other PCNA partners binding to the interconnecting loop domain of PCNA include for example DNA polymerases, Clamp loader (Rfc1, Rfc3), Flpa-endonuclease (FEN-1), DNA ligase-1, topoisomerase II alpha, replication licensing factor (Cdt1), helicases and ATPases (Rrm3, WRN, RECQ5), mismatch repair enzymes (UNG2, MPG, hMYH, APE2), nucleotide excision repair enzyme (XPG), histone chaperone (CAF-1), poly(ADP-ribose) polymerase (PARP-1), chromatin remodelling factor (WSTF), DNA methyltransferase (DNMT1), sister-chromatid cohesion factors (Eco1™, Chl1), cell cycle regulators (p57), and apoptosis regulators (ING1b, p53). The polypeptide liable to bind PCNA can thus correspond to any of those proteins.

More specifically, the inventors have found that cytoplasmic PCNA of neutrophil cells is liable to interact with carboxyp21 (see example 6) and with several procaspases including procaspase-8, procaspase-10, procaspase-3 and procaspase-9 (see example 8).

By “inhibiting an interaction” is meant preventing the binding of a molecule to another one. The inhibition of an interaction may be measured by various methods well-known by one skilled in the art. For instance, it may be measured by western blot assays, ELISA, co-immunoprecipitation (co-ip) assays, pull-down assays, crosslinking assays, label transfer approaches (FRET or HTRF assays) or yeast two-hybrid assays. The skilled in the art can easily determine if a compound inhibits an interaction between PCNA and a polypeptide liable to bind to PCNA such as p21, carboxyp21 or a procaspase by carrying out a competitive binding assay.

In the frame of the present invention, the compound preferably inhibits an interaction between PCNA and at least one polypeptide liable to bind to PCNA at the interdomain connecting loop of PCNA, i.e., the region of the PCNA protein spanning from the L121 residue to the E132 residue of SEQ ID NO: 1. Indeed, the inventors have found that peptides inhibiting an interaction taking place at this domain (i.e. peptides 3, 4 and carboxyp21) trigger apoptosis of neutrophils. The skilled in the art can easily determine if a compound inhibits an interaction taking place at the at the interdomain connecting loop of PCNA e.g. by carrying out a competitive binding assay with carboxyp21, which is known to bind to the interdomain connecting loop of PCNA.

As used herein, the term “p21” refers to the human p21 protein, also called p21/Waf1/Cip1, CAP20, CDKN1, CIP1, MDA-6, p21CIP1, SDI1 or WAF1. In a preferred embodiment, “p21” refers to a protein of sequence SEQ ID NO: 26. However, this term also encompasses allelic variants and splice variants of the protein of SEQ ID NO: 26.

Throughout the present specification, a “disease involving an inflammatory process” refers to a disease due to an exacerbated immune response called inflammation. Inflammation is for example characterized by the following features: redness, heat, swelling, and pain. The exacerbated inflammation may for instance be caused by infections by pathogens, burns, chemical irritants, frostbite, toxins, physical injuries, immune reactions due to hypersensitivity, autoimmune processes, autoinflammatory processes, ionizing radiations, foreign bodies, graft rejection or ischemia. In the frame of the present specification, a disease involving an inflammatory process is also called an inflammatory disease.

In a preferred embodiment, the disease involving an inflammatory process is mediated by neutrophil cells. The invention thus concern a compound inhibiting an interaction between PCNA and at least one polypeptide liable to bind to PCNA, for use in inducing neutrophil apoptosis and/or in the treatment of a disease involving an inflammatory process mediated by neutrophil cells.

More preferably, the disease involving an inflammatory process is a cardiac pathology, a vascular pathology, a pulmonary pathology, a digestive pathology, a cutaneous pathology, or a kidney pathology (caused or not by an infection).

Still more preferably, the disease involving an inflammatory process (i.e. the inflammatory disease) is selected from the group consisting of a cardiac ischemia (especially at day 4 after a stroke), a bacterial endocarditis, a purulent pericarditis (for example associated with bacterial endocarditis, with pneumonia, or with mediastinal infections), a polyarteritis nodosa, a Kawasaki disease, a leucytoclastic vasculitis, a microscopic polyangiitis, a bacillary angiomatosis (for example associated with AIDS), an adult respiratory distress syndrome, a cystic fibrosis, a bacterial, viral or mycoplasmal bronchopneumonia, an ulcer, a Crohn's disease, burns, gout (for example acute gout), pseudogout, arthritis (for example infectious arthritis, rheumatoid polyarthritis, rheumatoid arthritis, juvenile arthritis, or osteoarthritis), seronegative spondyloarthritides, transplant rejection, myocarditis, amyloidosis, Horton disease, Takayasu disease, cryoglobulinemia vasculitis, purpura rheumatoid, ANCA-positive vasculitis (Wegener granulomatosis, Churg-Strauss syndrome), atheroma, chronic obstructive pulmonary disease, pan-lobular emphysema, interstitial pneumonitis, ulcerative colitis, celiac disease, bullous dermatitis, neutrophilic dermatitis, Still disease, micro-crystallin arthropathies (gout, articular chondrocalcinosis, deposits of hydroxyapatite), psoriatic arthritis, coxopathy, systemic lupus, inflammatory myopathies, necrotizing myopathy, glomerulonephritis, interstitial nephropathy, granulomatous nephropathy, uveitis.

According to the invention, the expression “inflammatory cells” refers to leukocytes such as granulocytes (neutrophils, basophils, eosinophils), monocytes, macrophages, dendritic cells or lymphocytes. Preferably, the expression “inflammatory cells” refers to neutrophils.

In a preferred embodiment, the compound for use in the treatment of diseases involving an inflammatory process induces apoptosis of inflammatory cells. More preferably, said compound induces apoptosis of neutrophil cells.

Determining whether a compound induced apoptosis of inflammatory cells may be measured by various methods well-known by one skilled in the art. For instance, it may be quantified by measuring the amount of externalized phosphatidylserine, e.g. after annexin-V labeling. In such an experiment, externalized phosphatidylserine may be stained with a fluorochrome-coupled annexin V, thus allowing detection of apoptotic cells by flow cytometry, as described in Example 1.

The term “treatment” is understood to mean treatment for a curative purpose (aimed at least at alleviating or stopping the development of the pathology) or for a prophylactic purpose (aimed at reducing the risk of the pathology appearing).

Peptides for Use in the Treatment of Neutropenia

In another aspect, the present invention pertains to a peptide comprising the carboxy-terminal region of PCNA, or a fragment of at least six consecutive amino acids thereof, for use in the treatment of neutropenia, said carboxy-terminal region of PCNA being defined as the region consisting of amino acids 249 to 261 of SEQ ID NO: 1.

In a preferred embodiment, the peptide comprises or consists of a fragment of at least 6, 10, 15 or 20 consecutive amino acids of the carboxy terminal region of PCNA (amino acids 249-261 of SEQ ID NO: 1). Indeed, as shown in Example 9, peptides 8 and 9, which comprise a PCNA fragment of SEQ ID NO: 7, are capable of preventing neutrophil apoptosis. Thus, the peptide may for example comprise or consist of a sequence SEQ ID NO: 7, or a sequence at least 80, 85, 90 or 95% identical thereto, or a fragment of at least 6, 10, 15 or 20 consecutive amino acids thereof.

The peptide according to the invention may further comprise a tag, e.g. a tag enhancing entry of the peptide into cells. The tag may for example comprise or consist of a tag derived from the HIV-1 Tat polypeptide (preferably located at the N-terminal extremity of the peptide) or a RYIRS tag (preferably located at the C-terminal extremity of the peptide). SEQ ID Nos. 16 and 17 are specific examples of peptides according to the invention consisting of a fragment of at least 6 consecutive amino acids of the carboxy terminal region of PCNA fused to a tag.

The peptide may further comprise chemical modifications, such as e.g. acetylation, in order to enhance its solubility or biodisponibility.

Therefore, the invention pertains to a peptide, preferably a peptide comprising or consisting of:
a) an amino acid sequence consisting of SEQ ID NO: 7;
b) an amino acid sequence consisting of SEQ ID NO: 7 fused to a tag;
c) an amino acid sequence consisting of SEQ ID NO: 16 or SEQ ID NO: 17;
d) a sequence homologous to the sequence of any one of (a) to (c) and presenting at least 80% of identity with said sequence of any one of (a) to (c); and
e) an amino acid sequence consisting of a fragment of at least 6 consecutive amino acids of the sequence of any one of (a) to (c). for use in the treatment of neutropenia.

In a preferred embodiment, the peptide for use in the treatment of neutropenia prevents and/or inhibits apoptosis of inflammatory cells, preferably of neutrophil cells. Determining whether a compound induced apoptosis of inflammatory cells may be measured by various methods well-known by one skilled in the art. For instance, it may be quantified by measuring the amount of externalized phosphatidylserine, e.g. after annexin-V labeling. In such an experiment, externalized phosphatidylserine may be stained with a fluorochrome-coupled annexin V, thus allowing detection of apoptotic cells by flow cytometry, as described in Example 1.

The term “neutropenia” refers to a hematological disorder characterized by an abnormally low number of neutrophils, for example under 1500 neutrophils/μL of blood, preferably under 1000 neutrophils/μL of blood, and most preferably under 500 neutrophils/μL of blood.

As used herein, this term includes chronic, cyclic and acute neutropenia. The neutropenia may for example correspond to a chronic idiopathic neutropenia, a congenital neutropenia or a secondary neutropenia such as an infection-induced neutropenia, a drug-induced neutropenia, an alcoholism-induced neutropenia, an autoimmune neutropenia, a chronic secondary neutropenia in AIDS, a neutropenia caused by bone marrow replacement, a neutropenia caused by cytotoxic chemotherapy, a neutropenia caused by radiation therapy, a neutropenia caused by folate or vitamin B12 deficiency, a neutropenia caused by hypersplenism, or a neutropenia caused by T γ-lymphoproliferative disease.

Peptides According to the Invention

Another aspect of the invention pertains to a peptide as defined in the above paragraphs.

Preferably, the peptide according to the invention is derived from the amino acid sequence of the PCNA protein.

In a preferred embodiment, the peptide according to the invention has an amino acid sequence selected from the group consisting of:
a) an amino acid sequence consisting of SEQ ID NO: 3 or 7;
b) an amino acid sequence consisting of SEQ ID NO: 3 or 7 fused to a tag;
c) an amino acid sequence consisting of SEQ ID NO: 11; SEQ ID NO: 12; SEQ ID NO: 16 or SEQ ID NO: 17 and
d) a sequence homologous to the sequence of any one of (a) to (c) and presenting at least 80% of identity with said sequence of any one of (a) to (c).
e) an amino acid sequence consisting of a fragment of at least 6, 10, 15 or 20 consecutive amino acids of the sequence of any one of (a) to (c).

When the peptide is derived from another protein than PCNA, the peptide according to the invention is preferably liable to bind to PCNA, in particular to cytoplasmic PCNA found in neutrophil cells.

In addition, the peptide according to the invention the peptide preferably modulates (i.e. induces, prevents or inhibits) apoptosis of inflammatory cells, preferably of neutrophil cells. For instance, peptides comprising or consisting of a sequence of SEQ ID NO: 3 and peptides derived therefrom preferably induce apoptosis of inflammatory cells. On the other hand, peptides comprising or consisting of a sequence of SEQ ID NO: 7 and peptides derived therefrom preferably prevent and/or inhibit apoptosis of inflammatory cells.

Pharmaceutical Compositions According to the Invention

The invention further pertains to a pharmaceutical composition comprising a peptide as defined herein and one or more physiologically acceptable carriers.

Pharmaceutical compositions comprising a peptide of the invention include all compositions wherein the peptide is contained in an amount effective to achieve the intended purpose. In addition, the pharmaceutical compositions may contain suitable physiologically acceptable carriers comprising excipients and auxiliaries which facilitate processing of the active compounds into preparations which can be used pharmaceutically.

The term “physiologically acceptable carrier” is meant to encompass any carrier, which does not interfere with the effectiveness of the biological activity of the active ingredient and that is not toxic to the host to which is administered. Suitable physiologically acceptable carriers are well known in the art and are described for example in Remington's Pharmaceutical Sciences (Mack Publishing Company, Easton, USA, 1985), which is a standard reference text in this field. For example, for parenteral administration, the above active ingredients may be formulated in unit dosage form for injection in vehicles such as saline, dextrose solution, serum albumin and Ringer's solution.

Besides the physiologically acceptable carrier, the compositions of the invention can also comprise minor amounts of additives, such as stabilizers, excipients, buffers and preservatives. The composition of the invention may further comprise a second active principle.

The peptide of the present invention may be administered by any means that achieve the intended purpose. For example, administration may be achieved by a number of different routes including, but not limited to subcutaneous, intravenous, intradermal, intramuscular, intraperitoneal, intracerebral, intrathecal, intranasal, oral, rectal, transdermal, buccal, topical, local, inhalant or subcutaneous use. Parenteral and topical routes are particularly preferred.

Dosages to be administered depend on individual needs, on the desired effect and the chosen route of administration. It is understood that the dosage administered will be dependent upon the age, sex, health, and weight of the recipient, concurrent treatment, if any, frequency of treatment, and the nature of the effect desired. The total dose required for each treatment may be administered by multiple doses or in a single dose.

Depending on the intended route of delivery, the compounds may be formulated as liquid (e.g., solutions, suspensions), solid (e.g., pills, tablets, suppositories) or semisolid (e.g., creams, gels) forms.

The invention also pertains to a method of treating a disease involving an inflammatory process or neutropenia comprising the step of administering an effective amount of a peptide as defined herein to a subject in need thereof.

By “effective amount” is meant an amount sufficient to achieve a concentration of peptide which is capable of preventing, treating or slowing down the disease to be treated. Such concentrations can be routinely determined by those of skilled in the art. The amount of the compound actually administered will typically be determined by a physician or a veterinarian, in the light of the relevant circumstances, including the condition to be treated, the chosen route of administration, the actual compound administered, the age, weight, and response of the subject, the severity of the subject's symptoms, and the like. It will also be appreciated by those of skilled in the art that the dosage may be dependent on the stability of the administered peptide.

By “subject in need thereof” is meant an individual suffering from or susceptible of suffering from the disease involving an inflammatory process or the neutropenia to be treated. The individuals to be treated in the frame of the invention are preferably human beings.

Method of Screening According to the Invention

The inventors have found that PCNA, in particular cytoplasmic PCNA, plays a role in modulating apoptosis in inflammatory cells such as neutrophils.

The invention thus pertains to the in vitro use of PCNA as a target for screening for drugs for the treatment of neutropenia or diseases involving an inflammatory process.

More particularly, the invention pertains to a method for the identification of a compound for use in the treatment of neutropenia or diseases involving an inflammatory process, comprising:
a) contacting a candidate compound with PCNA and at least one polypeptide liable to bind to PCNA;
b) comparing the quantity of PCNA bound to said at least one polypeptide liable to bind to PCNA in the presence and in the absence of the candidate compound; and
c) selecting the candidate compound if the quantity of PCNA bound to said at least one polypeptide liable to bind to PCNA is lower in the presence of said candidate compound than in the absence of said candidate compound.

In this method, PCNA preferably corresponds to cytoplasmic PCNA. Most preferably, PCNA corresponds to cytoplasmic PCNA obtained from neutrophil cells.

Comparing the quantity of PCNA bound to said at least one polypeptide liable to bind to PCNA in the presence and in the absence of the candidate compound can be done by various methods well-known to one skilled in the art. For instance, this can be done by co-immunoprecipitation and western blot assays, as described in Example 1, or by a yeast two-hybrid screening assay, or by flow-cytometry (e.g. using a BIAcore system).

In the frame of this method, carboxyp21 may for example be used as the polypeptide liable to bind to PCNA. Indeed, carboxyp21 is known to interfere with PCNA partners in proliferating cell (Warbrick et al. 2006; Oncogene 25:2850-2859). In addition, as detailed in Example 6, carboxyp21 is capable to interfere with PCNA in neutrophil cells since it is capable of countering PCNA-mediated neutrophil anti-apoptotic effect. Therefore, displacement of carboxy-21 by the candidate compound in a competitive binding assay indicates that the candidate compound also binds to PCNA at the interdomain connecting loop of PCNA, and can therefore be used in the treatment of inflammatory diseases.

Alternatively, peptide 3 (SEQ ID NO: 11), peptide 4 (SEQ ID NO: 12) or peptide 8 (SEQ ID NO: 16) may also be used as polypeptides liable to bind to PCNA in the frame of the method according to the invention. In particular, carboxyp21, peptide 3 and peptide 4 may be used when screening for drugs for the treatment of inflammatory diseases, and peptide 8 may be used when screening for drugs for the treatment of neutropenia.

The compounds and drugs screened in the frame of the present invention include but are not limited to peptides, small molecules, antibodies, aptamers and nucleic acids such as antisense nucleic acids and siRNAs.

All references cited herein, including journal articles or abstracts, published patent applications, issued patents or any other references, are entirely incorporated by reference herein, including all data, tables, figures and text presented in the cited references.

The invention will be further evaluated in view of the following examples and figures.

### BRIEF DESCRIPTION OF THE FIGURES

FIG. 1. PCNA is expressed exclusively in the cytosol of mature neutrophils. (A) PCNA immunodetection in neutrophils (PMN), lymphocytes (Ly) or PLB985 promyelocytic cells. 50,000 cells/lane were analyzed using PC10 mAb as the primary Ab. (B) PCNA expression in different neutrophil subcellular compartments with b-actin (57 kDa), human neutrophil elastase (29 kDa) or lamin B (42 kDa) serving as control markers for the cytosolic (Cyt), granular (Gr) and nuclear (Nu) fractions, respectively. The SDS-PAGE gel was run using 50 μg of proteins/lane and PCNA was detected with the PC10 mAb. (C) Immunofluorescence analysis of PCNA localization in human CD34⁺cells during in vitro neutrophil differentiation. PCNA expression was examined before (CD34⁺) and at different times (7 or 13 days) during CD34⁺-granulocyte differentiation, and in mature neutrophils, using the rabbit pAb Ab5 and TO-PRO3 Iodide for nuclear labeling. Percentages of cells exhibiting either strict nuclear (white bars) or a mixted nuclear-cytoplasmic localization (hatched bars) or a strict cytoplasmic (black bars) were obtained by counting the cells under the microscope. The percentages were then plotted on the histograms displayed at the right of each panel. Panels A, B and C show representative experiments out of three yielding the same results.

FIG. 2. PCNA is degraded by the proteasome during neurophil apoptosis. (A) PCNA-expression kinetics in neutrophils incubated at 37° C. for 1, 3 or 15 h, alone (constitutive apoptosis) or with anti-Fas mAb (10 ng/ml) or gliotoxin (2 mg/ml). Western-blot analysis of neutrophil cytosolic fractions (50 μg/lane) using the anti-PCNA PC10 mAb. Actin immunoblotting served as a loading control on the same membrane. (B) Flow cytometric measurement of phosphatidylserine externalization on neutrophils cultured as in (A) and labeled with annexin-V-FITC and 7-AAD to assess apoptosis and necrosis, respectively. (C, D) Spectrophotometric determination of caspase-8 (C) and caspase-3 (D) activities in neutrophil lysates using their respective specific chromogenic IETD-pNA and DEVD-pNA substrates. (E) Effect of the proteasome inhibitor PS-341 at the indicated concentrations, on survival (upper panel) and on PCNA expression in neutrophils (lower panel), cultured as in (A). Apoptosis was obtained by incubating neutrophils at 37° C. for 15 h (T₁₅ₕ) and neutrophil survival was evaluated as the percentage of annexin-V⁻ 7-AAD⁻ neutrophils, to exclude cell apoptosis and necrosis, and compared with freshly isolated neutrophils (Tₒ). Values are means±SEM. of four independent experiments performed in duplicate, P*<0.05 (Student's t test). A representative PCNA immunoblot obtained under the same experimental settings is shown on the lower panel (F) Effect of PS-341 (1 mM) on PCNA expression in neutrophils incubated for 6 h (T₆ₕ) at 37° C. with anti-Fas or gliotoxin and compared with freshly isolated neutrophils (T₀). In the lower panel, colloidal gold staining of the membrane was used as a loading control. Panels A-D, F are from representative experiments that were performed at least four times and yielding identical results.

FIG. 3. Stable PCNA protein levels are maintained in neutrophils exposed to G-CSF, in vitro or in vivo. (A) Time-dependent modulation of PCNA-protein expression by G-CSF. Neutrophils were cultured with or without G-CSF (1000 U/ml) at 37° C. After 0, 3, 6 or 15 h of incubation, cells were lysed and Western-blot analysis was performed with the PC10 mAb, while anti-actin served as the loading control. (B) G-CSF dose-dependent effect on PCNA-protein expression. Neutrophils were incubated for 15 h at 37° C. in the absence (0) or in the presence of G-CSF (200 or 1000 U/ml) and analyzed as described in (A). The percentages of viable neutrophils i.e., annexin-V⁻ 7-AAD⁻ cells under the same conditions are reported in the histogram below the blot. (C) Kinetic analysis of PCNA and BLyS mRNA expressions and transcriptions in G-CSF-treated neutrophils. Total RNA was extracted from neutrophils cultured with or without 1000 U/ml G-CSF for the times indicated and analyzed for PCNA, BLyS and □₂-microglobulin (β2m) gene expressions by real-time RT-PCR and primary transcript real-time RT-PCR. Their expression is given as mean normalized expression (MNE) units after normalization to β2m of triplicate reactions for each sample. For a,b,c, data are from one experiment representative out of three. (D) Analysis of PCNA-protein expression in neutrophils isolated from G-CSF-treated donors. Neutrophils from a representative G-CSF-treated donor were analyzed either before or during (+G-CSF) cytokine exposure, with PCNA immunodetected in lysates from either freshly isolated (0) or 15-h-cultured cells. The percentages of viable neutrophils after culture as in (B) are shown on the histogram below the blot. (E) Confocal immunofluorescence microscopy of neutrophil PCNA detected with the Ab5 pAb. Neutrophils were isolated from a donor before or during (+G-CSF) in vivo G-CSF treatment. Data are from one G-CSF-treated donor, representative of four different donors.

FIG. 4. PCNA expression in murine neutrophils isolated from BM or from BALF of mice administrated with LPS intranasally. (A) Confocal immunofluorescence microscopy of neutrophil PCNA detected with the Ab5 pAb in mouse neutrophils isolated from BM or from peripheral blood (B) Analysis of PCNA-protein expression in BM-isolated mouse neutrophils treated with increasing gliotoxin concentrations. Western blot analysis of procaspase-3 was performed on neutrophil cytosolic fractions (20 μg/lane) and colloidal gold staining of the membrane was used as loading control. The percentages of viable neutrophils measured after annexin-V labelling are shown on the upper histogram. (C) Kinetics of neutrophil and macrophage recruitment within the airways following intranasal LPS instillation. BALF were obtained 16, 24, 48, and 72 h after LPS challenge and stained by MGG to discriminate between neutrophils and macrophages as illustrated in the lower panel below (D) Confocal immunofluorescence microscopy of neutrophil isolated from BALF after 16 h or 48 h LPS challenge. (E) PCNA and procaspase-3 Western blot analysis in neutrophil cytosolic fractions (50 μg/lane) isolated from BM and BALF as described in (B). (F) PCNA Apoptosis in BM and BALF neutrophils (PMN) was assessed after an overnight incubation at 37° C. by annexin-V labeling.

FIG. 5. Potentiation of neutrophil apoptosis by carboxyp21, a PCNA-competing peptide. The synthetic carboxyp21 and a control modified p21-peptide (whose charged amino acids, identified as crucial for the binding to PCNA, were modified to prevent its binding to PCNA) were incubated with neutrophils to evaluate their effect on apoptosis. (A) Structure of PCNA (purple, cyan and orange are used to distinguish the three monomers) bound to two carboxyp21 (green) and one modified p21-peptide (red). The structure represented is obtained at the end of the MD simulation. The secondary structure elements are highlighted by a ribbon representation; two different views are presented (from above the ring and from the side). While the carboxyp21 remains strongly bound to PCNA, the interactions of the modified peptide with PCNA are lost rapidly thus demonstrating a lower affinity for PCNA. (B, C) Exposure to carboxyp21 enhanced constitutive neutrophil apoptosis. Neutrophils were cultured for 6 h at 37° C. alone or with 50 mM of carboxyp21 or modified carboxyp21. The percentages of apoptotic neutrophils were assessed as depolarized mitochondria after DiOC₆ labeling (B) or phosphatidylserine externalization after annexin-V labeling (c). Basal apoptosis was assessed before incubation. Data are means±SEM of five independent experiments, P*<0.05; P<0.01 (Student's t test). (D) Decreased PCNA expression in neutrophils exposed to increasing carboxyp21 concentrations for 3 h paralleled apoptosis. Apoptosis was measured by annexin-V labeling. In the same samples, PCNA expression was evaluated by Western blot analysis and the bands were quantified by densitometric scanning. Data are from one representative experiment out of four. (E) Carboxyp21 reversed G-CSF neutrophil-prosurvival effect. Neutrophils were incubated with or without G-CSF (1000 U/ml), in the presence or absence carboxyp21 (50 μM) for 15 h before determining the percentage of apoptotic cells by annexin-V labeling. Data are means±SEM of at least five independent experiments, *P<0.05; P**<0.01 (Student's t test).

FIG. 6. Stable PCNA transfection protects neutrophil-differentiated PLB985 myeloid cells from apoptosis. (A) PCNA expression in control (pcDNA3-transfected cells) and pcDNA3-PCNA-transfected PLB985 cells. PCNA was detected by immunofluorescence using the Ab5 pAb in control and stably transfected (PCNA) PLB985 cells, before (−DMF) and after DMF-induced differentiation (+DMF). Fluorescence was quantified by Image J software before differentiation (histogram). Data are means±SEM of four independent experiments, P**<0.01 (Student's t test). (B, C, D, E) DMF-differentiated control, PCNA- or mutated PCNA-transfected PLB985 cells were incubated with or without 2 μg/ml gliotoxin or 10 ng/ml TRAIL to induce apoptosis. (B) Percentage of cells in the sub-G1 phase showing DNA fragmentation after propidium iodide labeling. (C) Percentage of cells showing chromatin condensation after Hoeschst labeling. (D) Percentage of cells with mitochondrial depolarization after DiOC₆ labeling. (E) Percentage of cells with caspase-8 activation using a fluorescent IETD-based substrate. Data are means±SEM of four independent experiments (P**<0.05 and P*<0.01 using Student's t test).

FIG. 7. Co-immunoprecipitation (co-IP) experiments identify procaspase-8, procaspase-10 and pro-caspase-9 as PCNA partners. Co-IP experiments were performed using neutrophil cytosols. Unbound material (UB) and bound (B) immunoprecipitated proteins were analyzed by Western blot analysis. IP used PCNA pAb (IP PCNA) or empty beads (IP control), while Western-blot analysis used both PC10 anti-PCNA to ascertain the presence of PCNA and anti-procaspase-8 mAb (A) or anti-procaspase-10 mAb (B) or anti-procaspase-3 mAb (C) or anti-procaspase-9 mAb (D). (E) Kinetics of immunodetection of cleaved caspase-9 after an in vitro procaspase-9 activation assay performed using neutrophil cytosol, cytochrome c (50 μM) and ATP (1 mM), without or with recombinant PCNA (100 μM). (F) Caspase-9 activity in PLB985 cells overexpressing wild type or mutated PCNA as compared with controls. CD11b-caspase-9 double positive cells were measured by flow cytometry before and after DMF-induced differentiation. Data are means±SEM. of four independent experiments (P**<0.05 and P*<0.01 using Students t test).

FIG. 8. Knocking-down CDK2 or GADD45alpha expression by siRNA sensitizes DMF-differentiated PLB985 cells to apoptosis. DMF-differentiated PLB985 cells were transfected twice with control(CT)-siRNA or with CDK2-siRNA or with GAD45alpha-siRNA on days 3 and 4 after DMF treatment. Effect of CDK2 or GADD45alpha siRNA on the percentage of apoptotic PLB985 cells was measured by mitochondrial depolarization after DiOC₆ labeling. Data are means±SEM of 3 independent experiments.

FIG. 9. The nuclear SV40NLS-PCNA mutant has no anti-apoptotic effect. (A) CD11b membrane expression in neutrophil-differentiated PLB985 cells stably overexpressing wild type PCNA or the nuclear SV40NLS-PCNA mutant as compared with control PLB985. PLB985 cells were treated with DMF for 5 days to induce granulocyte differentiation. CD11b expression was measured by flow cytometry and expressed as the percentage of CD11b-positive cells. (B) Mitochondrial depolarization analysis after DIOC₆ labelling in PLB985 cells. Cells were incubated with or without 1 μg/ml gliotoxin for 16 h to induce apoptosis. The data are the percentage of apoptotic cells with a decreased mitochondria potential and are expressed as the mean±SEM of nine independent experiments, * p<0.05, (Wilcoxon's test). (C) Percentage of PLB985 cells in the subG1 phase showing DNA fragmentation after propidium iodide staining. Data are means±SEM of six independent experiments, P′<0.05 (Student's t test).

### BRIEF DESCRIPTION OF THE SEQUENCES

SEQ ID No. 1 shows the sequence of the PCNA polypeptide.

SEQ ID No. 2-7 show the sequence of peptides derived from the PCNA polypeptide.

SEQ ID No. 8 shows the sequence of the RYIRS tag.

SEQ ID Nos. 9-17 show the sequence of peptides derived from the PCNA polypeptide, either tagged at their C-terminal extremity with a RYIRS tag, or tagged at their N-terminal extremity with a tag derived from the HIV-1 Tat polypeptide. The peptides of SEQ ID Nos. 9-17 are referred to as peptide 1, 2, 3, 4, 5, 6, 7, 8 and 9, respectively.

SEQ ID Nos. 18-22 show the sequence of peptides mentioned in Example 1.

SEQ ID NO: 23 shows the sequence of a peptide derived from p21.

SEQ ID NO: 24 shows the sequence of a peptide derived from p21, tagged at its C-terminal extremity with a RYIRS tag. This peptide is referred to as carboxyp21.

SEQ ID NO: 25 shows the sequence of a peptide used a negative control.

SEQ ID NO: 26 shows the sequence of the p21 polypeptide.

SEQ ID NO: 27 shows the sequence of the TAT tag.

SEQ ID NO: 28 shows the sequence of an oligonucleotide used to construct the pcDNA3-NLS(SV40)-PCNA plasmid.

SEQ ID NO: 29 shows the sequence of an oligonucleotide used to construct the pcDNA3-NLS(SV40)-PCNA plasmid.

### EXAMPLES

### Example 1

### Materials and Methods

Neutrophil isolation, granulocytic differentiation of CD34⁺ precursors and cell culture. Human neutrophils from healthy (Etablissement Francais du Sang, Paris) or G-CSF-treated healthy donors (10 mg/kg for 5 days to induce hematopoietic stem cells mobilization) (Biotherapy Department, Necker Hospital, France) were isolated from EDTA-anticoagulated blood, using density-gradient centrifugation through polymorphoprep (Nycomed), as described in Witko-Sarsat et al. (1999, Blood 94:2487-2496). Blood donors gave their written informed consent to participate in this study, which was approved by the Inserm Institutional Review Board and Ethics Committee of Necker-Enfants Malades Hospital (Paris, France). Differentiation of CD34⁺ cells into granulocytes was induced as described in Hino et al. (2000, Br. J. Haematol. 109:314-321), with some minor modifications. Briefly, CD34⁺ cells were isolated from cord blood and then cultured with stem cell factor (SCF; 10 ng/ml), IL-3 (10 ng/ml) and IL-6 (100 ng/ml) for 7 days. CD36⁻ cells were isolated, and then incubated with G-CSF (10 ng/ml), SCF (100 ng/ml) and IL-3 (10 ng/ml) for 13 days to promote granulocyte differentiation. MGG staining at different times was used to monitor differentiation (data not shown).

HeLa, PLB985 and NB4 promyelocytic cell lines were cultured in RPMI supplemented with 10% fetal calf serum. NB4 cells were induced to differentiate with ATRA (1 mM) (Sigma) for 5 days and granulocyte differentiation was validated by CD11b expression and by morphological analysis after MGG staining (data not shown). PLB985 cell granulocyte differentiation was induced by exposure to 0.5% DMF for 5 days and validated as for NB4. Functional analysis of NADPH-oxidase activity was determined with lucigenin-amplified chemiluminescence in a single-photon luminometer (AutoLumat LB953, Berthold Co.). The chemiluminogenic substrate, lucigenin (10,10-dimethyl-9,9-biacridium dinitrate), was used to selectively measure NADPH-oxidase-dependent extracellular superoxide-anion formation. Briefly, 100 μl containing 5×10⁵ cells, neutrophils or PLB985 cells, were distributed into polystyrene tubes containing 100 μl of lucigenin (0.2 mM) and 50 μl of stimulus, either HBSS or PMA (16 μM final concentration). Luminescence was measured in duplicate over 40 min and expressed as integrated total counts.

Immunofluorescence labeling and confocal microscopy analysis. Immunolabeling of neutrophils, HeLa, CD34⁺, PLB985 or NB4 cells to study PCNA subcellular localization was done as described in Kantari et al. (2007, Blood 110:4086-4095). Cells were fixed in PBS-3.7% formaldehyde (Sigma) for 20 min on ice and permeabilized with Triton-X100 (0.25%) for 5 min at room temperature, followed by ice-cold methanol for 10 min, incubated with rabbit pAb diluted 1:25 (Ab5, Calbiochem), for 45 min, followed by biotinylated rabbit IgG, diluted 1:100 (Dako Cytomation) for 30 min, and then by streptavidin-coupled Alexa-555, diluted 1:200 (2 mg/ml, Molecular Probes) for 30 min. The nuclei were stained by TO-PRO3 Iodide (1 mM solution, Molecular Probes), diluted 1:100, for 30 min. For co-localization with PCNA, mouse anti-procaspase-8 (clone 12F5, 1:25 dilution, Alexis Biochemicals) or anti-procaspase-9 (clone P10-F7, 1:25 dilution, Santa Cruz) mAb were used as primary antibodies followed by an Alexafluor-488-conjugated anti-mouse mAb (Molecular probe, diluted 1:100). Slides were mounted using Permafluor mounting medium (Vector Laboratories) and analyzed by confocal microscopy with a Zeiss LSM-5 confocal scanning laser microscope. Fluorescence was quantified using Image J software version 1.42d (Becton-Dickinson).

Analysis of apoptosis. Neutrophil apoptosis was triggered by incubating neutrophils at 37° C. alone (constitutive apoptosis) or with anti-Fas mAb (10 ng/ml; Beckman-Coulter) or gliotoxin (2 mg/ml; Sigma) (Ward et al. 1999, J. Biol. Chem. 274:4309-4318) for the indicated times. Neutrophil apoptosis was evaluated by phosphatidylserine externalization after annexin-V and 7-AAD labeling (Kantari et al. 2007, Blood 110:4086-4095); mitochondrial depolarization after DiOC₆ labeling (Moriceau et al. 2009, J. Immuno1.182:7254-7263); caspase-3 and caspase-8 activities measured by spectrophotometry (Biovision) in neutrophil lysates. In DMF-differentiated-PLB985 cells, apoptosis was triggered by gliotoxin or by rTRAIL (10 ng/ml; RD System) (Yin et al. 2005, Int. J. Biochem. Cell Biol. 37:1696-1708) and caspase-8 and caspase-9 activities were determined after gating on CD11b⁺ cells using the Caspase-Glow® assay (Promega). Apoptosis-induced chromatin condensation and DNA fragmentation were assessed, respectively, after DNA staining with the blue fluorescent dye Hoechst (5 mg/ml) and propidium-iodide staining (Goepel et al. 2004, J. Leukoc. Biol. 75:836-843). Carboxyp21 (KRRQTSMTDFYHSKRRLIFSRYIRS, SEQ ID NO: 24) corresponding to the p21 carboxy-terminus sequence (residues 141-160) and containing a RYIRS carboxy-terminal extension to facilitate its cell entry was synthesized and purified (Genecust). A modified peptide in which the charged amino acids responsible for its binding to PCNA have been changed according to dynamic simulation studies (KRRQTGETDFDHAKAALIFSRYIRS, SEQ ID NO: 25) served as control. The effect of PS-341 (LC Laboratories) and MG132 were tested on neutrophil apoptosis and PCNA expression as indicated.

Real-time RT-PCR and primary transcript (PT) real-time RT-PCR. Real-time RT-PCR and PT real-time RT-PCR were performed as described in Tamassia et al. (2008, J. Immunol. 181:6563-6573) using gene-specific primer pairs (Invitrogen) available in the public database RTPrimerDB (world wide web site medgen.UGent.be/rtprimerdb/) under the following entry codes: human PCNA (7839), BLyS/TNFSF13B (7841) and α₂-microglobulin (β2m) (3534). The reaction conditions were identical for all primer sets, as follows: 50° C. for 2 min, 95° C. for 2 min, and then 40 cycles of 95° C. for 15 s and 60° C. for 1 min. β2m was selected as a normalizing gene, because of its stable expression levels in leukocytes. Data were calculated with Q-Gene software (world wide web site BioTechniques.com) and are expressed as mean expression (MNE) units after β2m normalization. PT real-time PCR was done as described in Rossato et al. (2007, Eur. J. Immunol. 37:3176-3189), using primers designed from an intron region of the selected genes (see public database RTPrimerDB) under the following entry codes: PT-PCNA (7840), PT-BLyS (7842) and β2m (3534). The same RNA samples were processed in the absence of reverse transcriptase and served as controls for genomic DNA contamination. Northern-Blot analysis was done as described in Tamassia et al. (2008, J. Immuno).181:6563-6573).

In vivo model of LPS-induced airway inflammation. Peripheral blood neutrophils and BM mature neutrophils from femurs were isolated using Percoll density gradient, as described in Mocsai et al. (2002, Immunity 16:547-558). Intranasal LPS administration in mice and collection of cells from BALF was performed as described in Chignard et al. (2000, Am. J. Physiol. Lung Cell Mol. Physiol. 279:L1083-1090). Mice were treated in accordance with Inserm guidelines in compliance with European animal-welfare regulations. Seven-week-old male C57B1/6 mice, weighing 25-30 g (Charles Rivers), were anesthetized by intra-muscular injection of a mixture of ketamine and xylasine, and then 330 μg/kg of LPS dissolved in 50 μl of saline were deposited intranasally. At different times, animals were killed by an intraperitoneal injection of a lethal dose of sodium pentobarbital (Sanofi) to isolate lung neutrophils. The trachea of each mouse was then cannulated and BAL performed with a syringe and multiple cycles of saline instillation (0.5 ml) and aspiration to obtain a BALF volume of 4 ml. The latter was centrifuged, pelleted cells were collected and differential cell counts made after cytospin centrifugation and staining with Diff-Quik products. BAL neutrophils were isolated by Ficoll density gradient centrifugation.

Plasmid vector construction and recombinant PCNA expression. Recombinant PCNA was produced in E. coli using the plasmid PETPCNA containing the human PCNA cDNA (kind gift from Dr Bruce Stillman, Cold Spring Harbor, N.Y.) and purified as described in Waga et al. (1994, Nature 369:574-578). To obtain PCNA overexpression in PLB985-cells, PCNA cDNA was subcloned into the expression vector pcDNA3/neo (Invitrogen) between HindIII and NotI restriction sites. Site-directed mutagenesis was performed, using the Quickchange method (Stratagene) to obtain PCNA mutated on charged amino acids within the known p21-interacting sequence located at the interdomain connecting loop (PCNA-D120A-D122A-E124A-E130A-E132A, positions of the mutations on SEQ ID

NO: 1). All cDNA sequences were confirmed by direct sequencing. PLB985 cells were transfected using the AMAXA system® device, according to the manufacturer's instructions. Briefly, cells (2×106) were resuspended in 100 μl of cell-line solution with 1 μg of plasmid (control pcDNA3-neomycin or pcDNA3-PCNA or pcDNA3-mutated PCNA). Cells were then electroporated and transferred into culture plates. Transfected cells were cloned and selected based on their resistance to neomycin (1 mg/ml).

Western blot analysis and co-immunoprecipitation experiments. Neutrophils were sonicated and cytosolic proteins were analyzed using either the PC10 mAb or the Ab5 pAb anti-PCNA according to standard immunoblot procedures (Moriceau et al. 2009, J. Immunol. 182:7254-7263). Co-immunoprecipitation experiments were performed using neutrophil cytosols obtained by cavitation (Witko-Sarsat et al. 1999, Blood 94:2487-2496). Cytosol (500 mg) was mixed with 50 ml of protein G and Ab5 pAb and incubated for 30 min at 4° C. under shaking. The sample was loaded onto a column containing Sepharose-coated magnetic beads (Milteny), washed with a stringent washing buffer (50 mM Tris, 500 mM NaCl, 1% NP-40, 0.5% Triton X-100). The column was washed twice with this buffer, twice with the same buffer except that the NaCl was 300 mM and, finally, 5 times with a hyposaline buffer (20 mM Tris pH 7.5). The sample was eluted from the column with 30 ml 5× sample buffer and analyzed by Western blotting using different mAb to potential partners. PCNA was detected on the Western blot with the PC10 mAb and procaspases were detected with either anti-procaspase-8 (Calbiochem) mAb or mouse anti-procaspase-10 (Santa Cruz) or anti-procaspase-9 (Santa Cruz) or anti-procaspase-3 (Santa Cruz) mAbs, followed by horseradish peroxidase-conjugated anti-mouse IgG (Nordic Immunology diluted 1:5000), using the SuperSignal West Pico detection kit (Pierce).

In silico design of the modified p21 peptide using dynamic simulations. To design a modified p21 peptide that does not bind PCNA and could be used as a control peptide, the inventors first inventoried at the atomic level of detail the PCNA-p21 interactions (data not shown) based on the trajectory obtained from 20 ns-long molecular dynamics (MD) simulations of PCNA complexed with three carboxyp21 peptides (RQTSMTDFYHSKR, SEQ ID NO: 18). The inventors then identified the p21 peptide amino acids to form strong interactions with PCNA and subsequently ran new MD simulations of PCNA complexed with the modified p21 peptides (RQTGETDFDHAKA, SEQ ID NO: 19). The initial set of coordinates used for the molecular dynamics (MD) simulations was prepared from the X-ray structure of PCNA complexed to the peptide SAVLQKKITDYFHPKK (PDB ID 1VYJ, SEQ ID NO: 20). Three simulations of the PCNA trimer were run: (i) MD1: PCNA trimer with a p21 peptide (RQTSMTDFYHSKR, SEQ ID NO: 21) interacting with each of the three monomers (labeled A, C and E in the X-ray file), (ii) MD2: PCNA with three modified peptides (RQTGETDFDHAKA, SEQ ID NO: 22) and (iii) MD3: PCNA with two p21 peptides (on PCNA monomers A and C) and one modified peptide (on PCNA monomer E). The simulation strategy was performed as previously described except that the production runs lasted for 20 ns. The systems contain ca. 100000 atoms including explicit water molecules, proteins and counter-ions. Calculations were run on 256 cores of a Cray XT4 using the NAMD software. Analysis of the obtained trajectories revealed the loss of strategic interactions between PCNA and the modified p21 peptide, providing evidence that it could barely bind PCNA. Interactions between the carboxyp21 and PCNA remained stable during the 20 ns of the simulations.

Statistical analysis. Statistical analysis was performed using the Statview software package. Comparisons were made using the student t test. Differences were considered significant when the P<0.05.

Peptide synthesis. Peptides of SEQ ID Nos. 9 to 17 were supplied by GeneCust. These peptides comprise a tag to facilitate entry of the peptide into the cell. The peptides were then diluted in PBS, or solubilised in ethanol or DMSO.

Effects of the peptides on neutrophil apoptosis. Neutrophils were isolated from the blood of healthy volunteers using polymorphoprep (Witko-Sarsat et al. 1999; Blood 94:2487-96). Physiological apoptosis of these neutrophils was then activated by incubating them at 37° C. during 16 h in RPMI culture medium complemented with 10% serum. Under these conditions, neutrophils undergo a spontaneous apoptosis which can be evaluated by measuring the phosphatidylserine externalization (Kantari et al. 2007; Blood 110:4086-95; Moriceau et al. 2009; J Immunol 182:7254-63). Phosphatidylserine was stained using a fluorochrome-coupled annexin V allowing detection of apoptotic neutrophils by flow cytometry using a Facscan (Beckton Dickinson). Neutrophils stained with annexin V express the phosphatidylserine and are recognized and phagocytosed by macrophages. Furthermore, a secondary staining with the 7AAD intercalating agent was used to indicate late apoptosis (Durant et al. 2004; J Leukocyte Biol 75:87-98). Thus, total apoptosis is measured by the percentage of cells being either 7-AAD or annexin-V-positive (the rest of the cells being considered as viable). Early apoptosis is measured by the percentages of neutrophils being both annexin-V positive and 7-AAD negative. Late apoptosis is measured by the percentages of neutrophils being both annexin-V positive and 7-AAD positive. The necrotic cells being 7AAD positive are excluded from the analysis on the basis of the data of size/granulometry by flow cytometry analysis. The tested peptides were added to the neutrophils culture at a final concentration of 200 μM.

Plasmid pcDNA3-NLS(SV40)-linker-PCNA construct. The plasmid pcDNA3-NLS(SV40)-linker-PCNA construct was constructed with pcDNA3-PCNA with 5′GGCCGCGCCATGCCGAAGAAGAAGCGCAAAGTAGGCGAAGGGCAAGGGCAAGGG CAAGGGCCGGGCCGCGGCTACGCGTATCGATCCC3′ and 5′TCGAGGGATCGATACGCGTAGCCGCGGCCCGGCCCTTGCCCTTGCCCTTGCCCTT CGCCTACTTTGCGCTTCTTCTTCGGCATGGCGC3′ oligonucleotides.

The flexible and hydrophilic linker sequence was from the pEVRF plasmid described in Leonhardt et al. 2000; J. Cell Biol. 149:271-280. The dimerized oligonucleotides was directly ligated with pcDNA3-PCNA digested by Not1 and Xho1.

### Example 2

### Mature Human Neutrophils Express PCNA Exclusively in their Cytoplasm

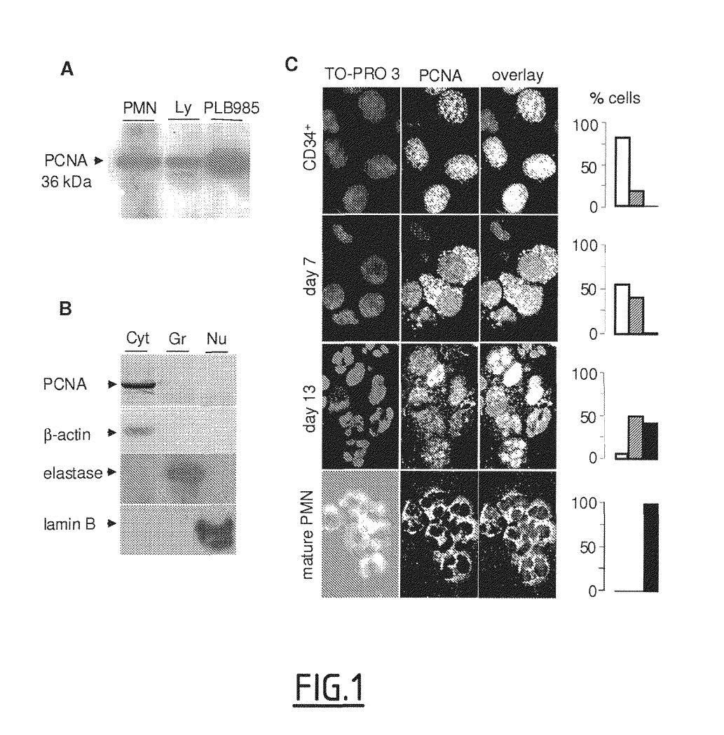

Western-blot analysis of neutrophil lysates readily detected PCNA in amounts comparable to those in lymphocytes but less than those in the PLB985 promyelocytic cell line (FIG. 1A).

Surprisingly, subcellular fractionation of neutrophils showed high PCNA contents only in the cytosol, and its absence in the nucleus and granules (FIG. 1B). The quality of the fractionation procedure was validated by the detection of specific markers: α-actin, elastase and lamin-B for cytosol, granules and nuclei, respectively.

### Example 3

### Nuclear-to-Cytoplasmic PCNA Relocalization Occurs During Granulocyte Differentiation

PCNA subcellular localization was also studied by confocal microscopy after PCNA immunolabeling during the course of in vitro granulocyte differentiation of human CD34⁺ cells, isolated from umbilical cord blood and cultured with IL-3 and G-CSF. Complete granulocyte maturation was evaluated by morphological analysis after May-Grünwald-Giemsa (MGG) staining. Before inducing differentiation, PCNA was detectable almost exclusively in the nucleus of CD34⁺ cells, whereas 7 days post IL3-G-CSF-treatment, the protein exhibited a mixed cytoplasmic and nuclear distribution (FIG. 1C). On day 13, most cells had multilobular nuclei with PCNA located in the cytoplasm, similar to what is observed in mature peripheral neutrophils.

Quantitative analysis (see histograms, FIG. 1C), consisting of counting the cells having a nuclear, cytoplasmic or mixed nuclear-cytoplasmic localization, confirmed this PCNA redistribution from the nucleus to the cytoplasm of differentiated neutrophils.

### Example 4

### PCNA is Targeted to Proteasomal Degradation During Neutrophil Apoptosis while it is Stabilized by G-CSF

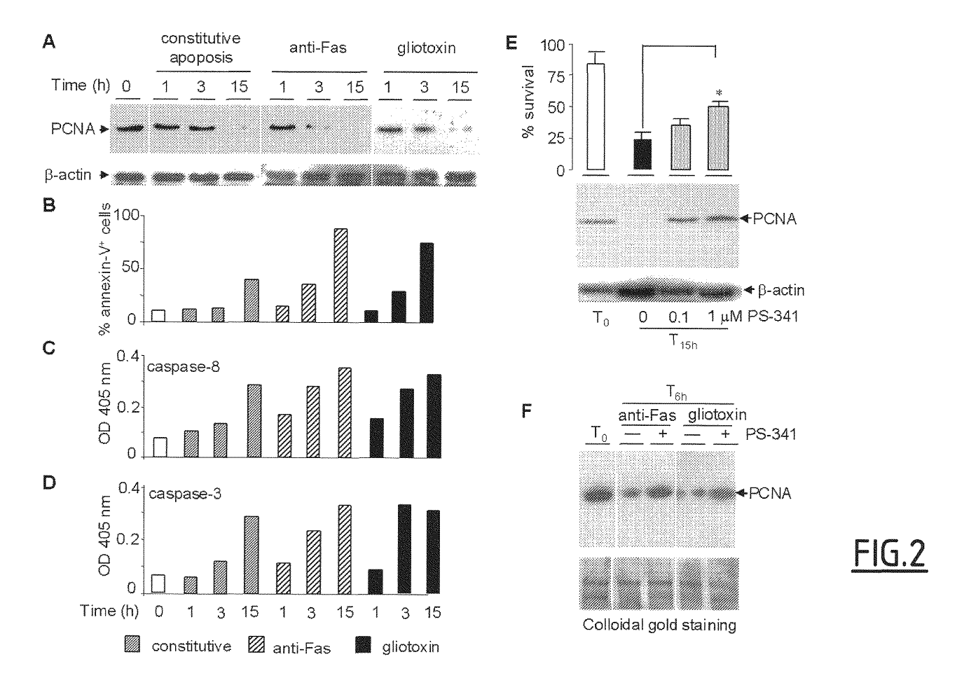

As indicated by its current name, PCNA levels are modulated during the cell cycle and are particularly high during the S-phase of proliferating cells. As shown below, PCNA expression also seemed to be modulated in non-proliferating neutrophils, however as a function of their survival status. Indeed, PCNA expression decreased in apoptotic neutrophils, independently of the death pathway, as it could be observed during constitutive or, more marked, after anti-Fas- or gliotoxin-potentiated apoptosis (FIG. 2A). The apoptotic process was evaluated by the phosphatidylserine externalization (FIG. 2B), which, as expected, confirmed a significant increase of annexin-V⁺ and 7-aminoactinomycin D (7-AAD)⁻ cells after a 15 h-incubation at 37° C., further enhanced by an anti-Fas mAb or gliotoxin. Similarly, time-dependent increases of caspase-8 (FIG. 2C) and caspase-3 (FIG. 2D) activities were observed in neutrophils during constitutive apoptosis, again further enhanced after anti-Fas or gliotoxin exposure. Finally, neutrophil apoptosis was also demonstrated by mitochondrial depolarization after DiOC₆ labeling. Thus, the decreased PCNA expression (FIG. 2A) appears to parallel the degree of apoptosis, regardless of the test or experimental conditions used. Notably, the proteasome inhibitors (PS-341) (FIG. 2E) and MG132 reversed the apoptosis-induced PCNA diminution and, consequentially, significantly inhibited neutrophil death. Interestingly, PS-341 also reversed PCNA degradation taking place after a 6 h-neutrophil incubation with anti-Fas or gliotoxin (FIG. 2F), thereby inferring that proteasome-mediated PCNA degradation occurs in the death-receptor and mitochondrial apoptotic pathways, respectively.

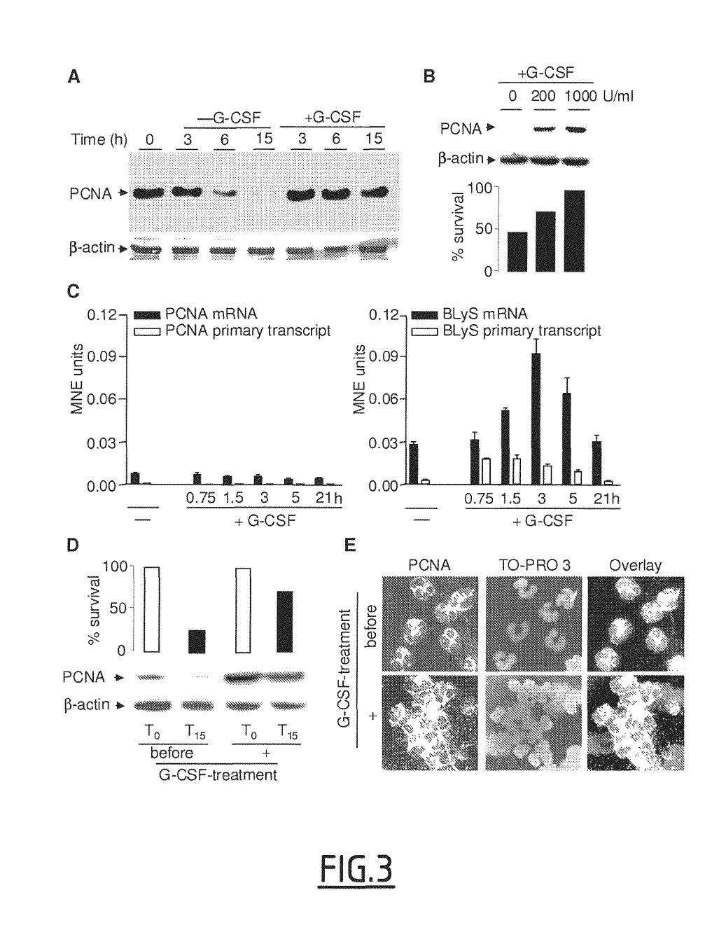

The observation that PCNA levels were tightly linked with neutrophil survival was corroborated by the stable PCNA levels maintained by G-CSF, which has been described as extending neutrophil longevity (Maianski et al. 2004; J. Immunol. 172:7024-7030). In fact, analysis of PCNA expression kinetics revealed that the decrease observed after a 6 h-incubation at 37° C. was totally prevented by G-CSF, as PCNA remained elevated until 15 h (FIG. 3A). Such G-CSF-induced PCNA level stabilization was dose-dependent and clearly associated with prolonged neutrophil survival (FIG. 3B). Incubating neutrophils with G-CSF for up to 21 h did not modify PCNA mRNA expression or transcription, as assessed by real-time RT-PCR and primary transcript real-time RT-PCR, respectively, but up-regulated BLyS gene expression (Scapini et al. 2003; J. Exp. Med. 197:297-302) (FIG. 3C). These observations were confirmed by using different pairs of PCNA-gene-specific primers and Northern-blotting experiments. Notably, interferon-g but not bacteria-derived formylated peptides, prevented PCNA degradation, again supporting its protective effect on neutrophil survival. PCNA involvement in positively regulating neutrophil survival was further confirmed by examining neutrophils isolated from the blood of healthy donors treated with G-CSF for 5 days. Indeed, the PCNA content in these neutrophils, assessed by immunoblotting, was markedly increased compared to that before G-CSF treatment (FIG. 3D).

This higher neutrophil cytoplasmic PCNA expression in G-CSF-treated donors, also confirmed by confocal microscopy analysis (FIG. 3E), was unequivocally associated with a decreased tendency of these neutrophils to undergo constitutive apoptosis (FIG. 3D).

### Example 5

### Low PCNA Levels in Murine Neutrophils Isolated During the Resolution of Lipopolysaccharide (LPS)-Induced Lung Inflammation

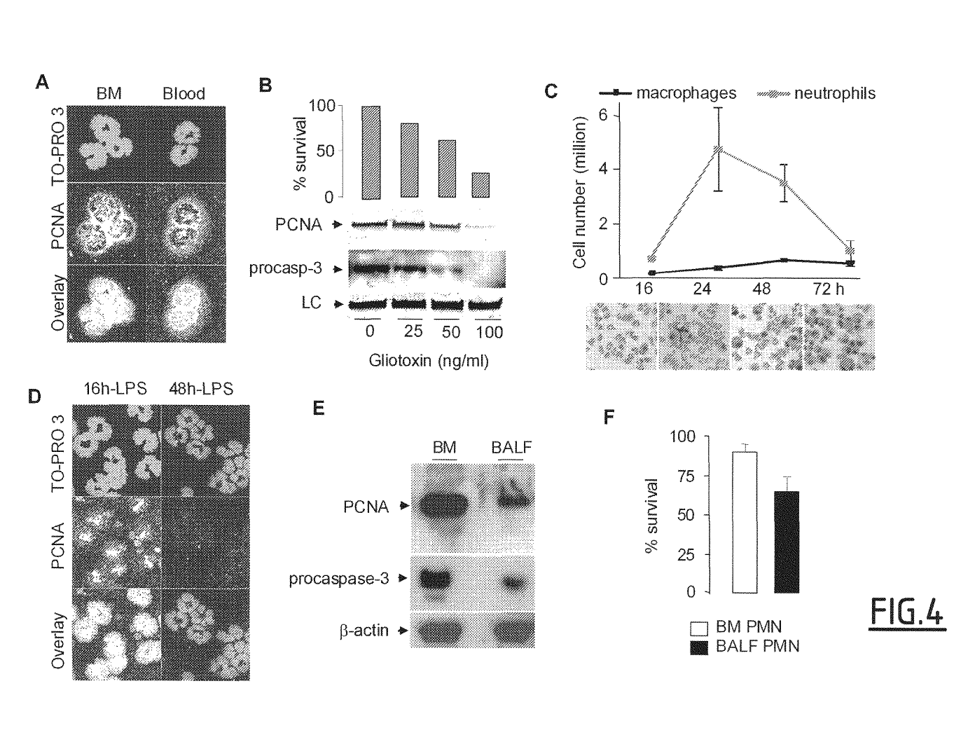

To gain more insight into the PCNA role in regulating neutrophil survival in vivo, the inventors studied mouse neutrophils isolated from either bone marrow (BM) or peripheral blood and observed that they expressed PCNA within their cytoplasm (FIG. 4A). In keeping with previous findings in human neutrophils, gliotoxin trigger apoptosis in BM neutrophils in a dose-dependent manner as assessed by annexin-V labelling (FIG. 4B). Pertinently, a parallel decrease in procaspase-3 and PCNA expressions were observed by Western blot analysis, thus confirming that PCNA was associated with mouse neutrophil survival. The inventors next switched to a murine LPS-induced lung-inflammation model that enables inflammatory neutrophil examination during inflammation resolution. As previously described (Chignard et al. 2000; Am. J. Physiol. Lung Cell Mol. Physiol. 279:L1083-1090), intranasal instillation of LPS triggers massive neutrophil recruitment within the airways, with maximal accumulation at 24 h (FIG. 4C). Inflammation resolution begins 48 h post-LPS administration and is characterized by decreased influx, increased apoptosis and macrophage-mediated phagocytosis of recruited neutrophils (FIG. 4C). Notably, neutrophil counts were very low after 72 h and, at 96 h, all the neutrophils appear to have been eliminated by macrophages (Chignard et al. 2000; Am. J. Physiol. Lung Cell Mol. Physiol. 279:L1083-1090). Indeed, lung neutrophils isolated from bronchoalveolar lavage fluids (BALF) 16 h post LPS-challenge expressed cytoplasmic PCNA whereas at 48 h post LPS-challenge, the expression of cytoplasmic PCNA was hardly detectable as shown by confocal microscopy analysis after PCNA immunolabelling (FIG. 4D). Western blot analysis of PCNA in neutrophils isolated from BM or from BALF 48 h post LPS-challenge confirmed the low PCNA expression in the latter (FIG. 4E). Interestingly, neutrophils isolated 48 h post LPS-challenge had a poor survival capacity, compared to BM neutrophils, as demonstrated by the increased percentage of annexin-V positivity after overnight culture at 37° C. (FIG. 4F). Again, the high BM-neutrophil survival rate was associated with markedly elevated PCNA levels. By contrast, lung neutrophils had sharply diminished PCNA and procaspase-3, thereby corroborating their proapoptotic state (FIG. 4E).

These data extend the in vitro findings and support the notion that PCNA, by regulating neutrophil viability, might act as a major player in controlling the neutrophil survival/apoptosis balance during the inflammation resolution.

### Example 6

### A p21 Peptide Known to Interfere with PCNA Partners Counters PCNA-Mediated Neutrophil Anti-Apoptotic Effects

PCNA has no intrinsic enzymatic activity and its biological role relies on its ability to mediate associations with different partners (Warbrick et al. 2000; Bioessays 22:997-1006, Maga et al. 2003; J. Cell Sci. 116:3051-3060). Most PCNA interactions occur at the interdomain connecting loop. The protein with the highest affinity for PCNA currently known is the CDK-inhibitor p21, which substantially jams the binding pocket, which explains its dominant role in inhibiting cell replication and other PCNA-regulated functions.

Therefore, the inventors tested the effect of a peptide corresponding to the residues 141-160 of p21 (so called carboxyp21), previously validated as able to interfere with PCNA partners in proliferating cell (Warbrick et al. 2006; Oncogene 25:2850-2859).

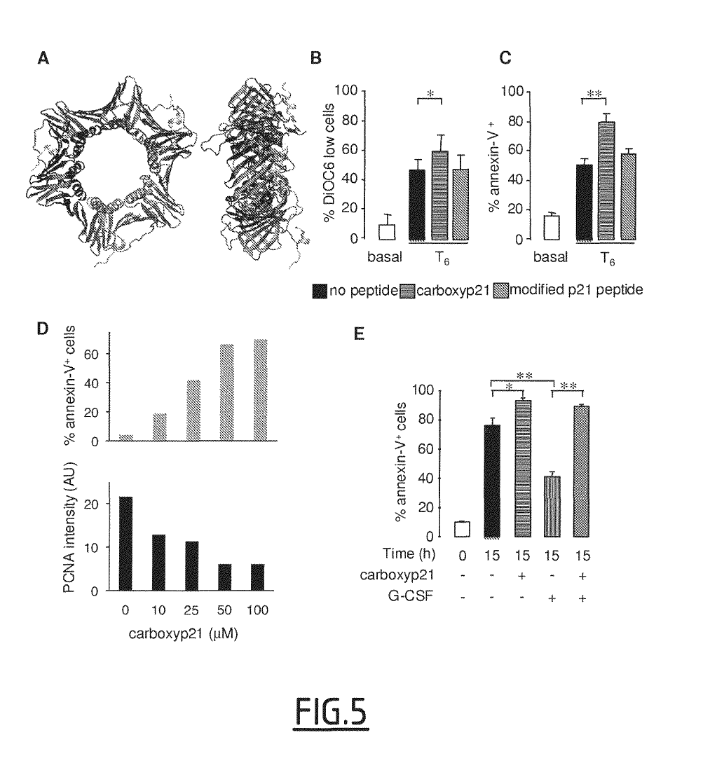

In mature neutrophils, p21 is barely detectable under basal conditions (Klausen et al. 2004; J. Leukoc. Biol. 75:569-578), thereby excluding its involvement in the regulation of cytosolic PCNA anti-apoptotic activity. A modified carboxyp21 peptide that does not bind PCNA was also designed using molecular dynamic simulations and used as control (FIG. 5A).

Carboxyp21, supposed to bind to PCNA and compete with PCNA partners, triggered neutrophil apoptosis as assessed by higher percentages of cells with depolarized mitochondria after DiOC₆ labeling, whereas no such activity was observed with the modified carboxy-21 peptide (FIG. 5B). The proapoptotic effect of carboxyp21 was also demonstrated by a significantly increased percentage of cells with externalized phosphatidylserine after annexin-V labeling (FIG. 5C). Note that carboxyp21-triggered apoptosis paralleled a dose-dependent decrease of PCNA expression (FIG. 5D). Moreover, carboxyp21 significantly inhibited G-CSF-induced neutrophil survival (FIG. 5E), once again strongly suggesting a PCNA regulatory role in neutrophil survival.

Based on these data, the inventors concluded that carboxyp21 triggered neutrophil apoptosis via a cell-cycle independent mechanism probably by competing with cytoplasmic PCNA partners.

### Example 7

### PCNA Overexpression Delays the Apoptosis Induction in Differentiated PLB985, a Cellular Model of Neutrophils

Neutrophils are short-lived, non-proliferating primary cells that are not suitable for transfection experiments. Therefore, promyelocytic cell lines, like PLB985, which can differentiate into neutrophils upon dimethyl-formamide (DMF) treatment, constitute valuable cell models mimicking mature neutrophils (Pedruzzi et al. 2002; Br. J. Haematol. 117:719-726.).

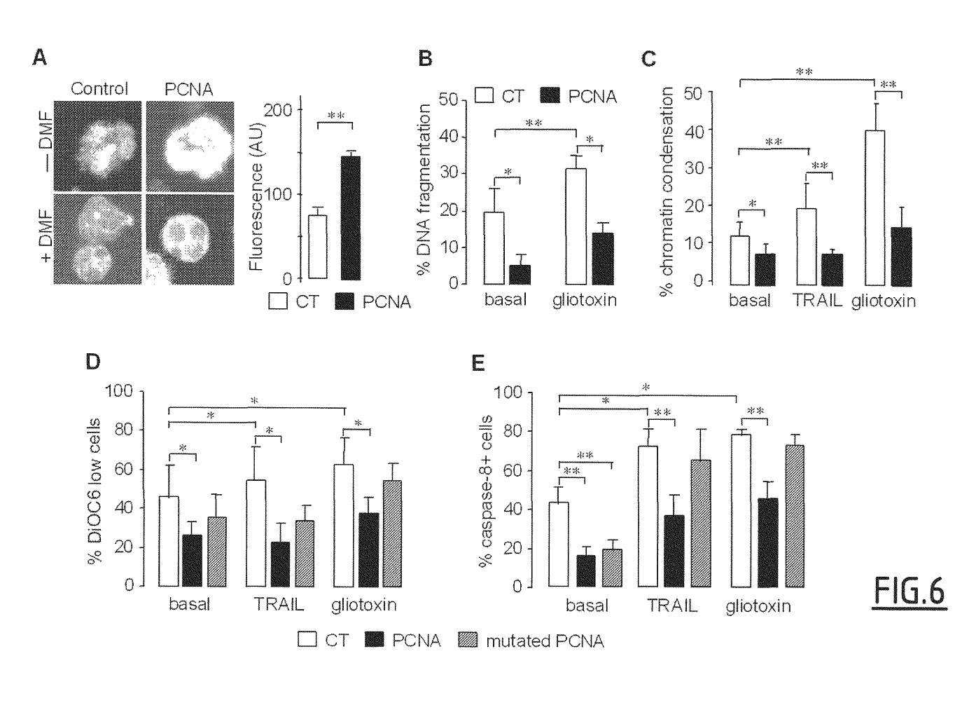

Accordingly, PLB985 cells were stably transfected with pcDNA3PCNA (PLB985-PCNA), as confirmed by confocal microscopy after PCNA immunolabeling before and after cell differentiation (FIG. 6A). Fluorescence quantification showed significantly increased PCNA expression in stably transfected PLB985-PCNA compared to control PLB985 stably transfected with the empty plasmid (FIG. 6A). Following DMF treatment, no difference in their CD11b expressions or NADPH-oxidase activities, respective phenotypic and functional markers of differentiated granulocytes, were observed. As expected, no superoxide production was obtained before differentiation, whereas a significant chemiluminescence was triggered by phorbol-12-Myristate-13 (PMA) both in control and PCNA-overexpressing PLB985. Hence, PCNA overexpression did not affect granulocyte differentiation and responsiveness. As previously described, apoptosis could be triggered in PLB985 cells either via the death receptor pathway using TRAIL or via the mitochondrial pathway using gliotoxin (Moriceau et al. 2009; J. Immunol. 182:7254-7263). After DMF-induced differentiation, PLB985 cells overexpressing PCNA were protected against TRAIL- or gliotoxin-induced apoptosis, compared to differentiated PLB985 transfected with the control plasmid, as judged by DNA fragmentation (FIG. 6B) or by chromatin condensation (FIG. 6C). In addition, either mitochondrial depolarization or caspase-8 activity were significantly lower in PLB985-PCNA after constitutive, TRAIL- or gliotoxin-induced apoptosis, compared to controls (FIG. 6D, E). Charge mutations in the PCNA interdomain connecting loop which is also the p21-interacting domain corresponding to residues 120-132 residues in the PCNA sequence were then used to determine if this PCNA domain was involved in its anti-apoptotic activity and PLB985 cells expressing an interdomain connecting loop-mutated PCNA were therefore generated. PCNA anti-apoptotic activity assessed by mitochondrial depolarization was significantly reduced in PLB985 expressing the interdomain connecting loop-mutated PCNA compared to PLB985-PCNA (FIG. 6D). Likewise, PLB985 stably transfected with this mutant PCNA also had increased caspase-8 activity compared to PLB985-PCNA after TRAIL- or gliotoxin-induced apoptosis (FIG. 6E). In the absence of apoptotic stimulus, PCNA protected against differentiation-induced apoptosis, which might reflect neutrophil physiological apoptosis.

Collectively, these data strongly suggest that some PCNA protein partner(s) could bind to the PCNA interdomain connecting loop to mediate the observed anti-apoptotic effect. These observations also corroborate the results showing that carboxyp21, by binding specifically to this PCNA site, triggered neutrophil apoptosis.

### Example 8

### PCNA Associates with Procaspase-8, Procaspase-10, Procaspase-3, Procaspase-9 and Interferes with Activation of the Latter One

Finally, the inventors performed co-immunoprecipitation (IP) experiments using neutrophil cytosols to identify potential cytosolic PCNA partners, which could participate to its anti-apoptotic activity.

Mcl-1 is one of the most important Bcl-2 homolog expressed in neutrophils, whose expression decreases during apoptosis. Because it was previously described as a PCNA partner in proliferating cells (Fujise et al. 2000; J. Biol. Chem. 275:39458-39465), the inventors investigated whether PCNA could bind Mcl-1. Although Mcl-1 could be successfully immunoprecipitated, no physical association was detected between PCNA and Mcl-1, or other Bcl-2 homologs, e.g., the pro-apoptotic proteins Bax or Bid.

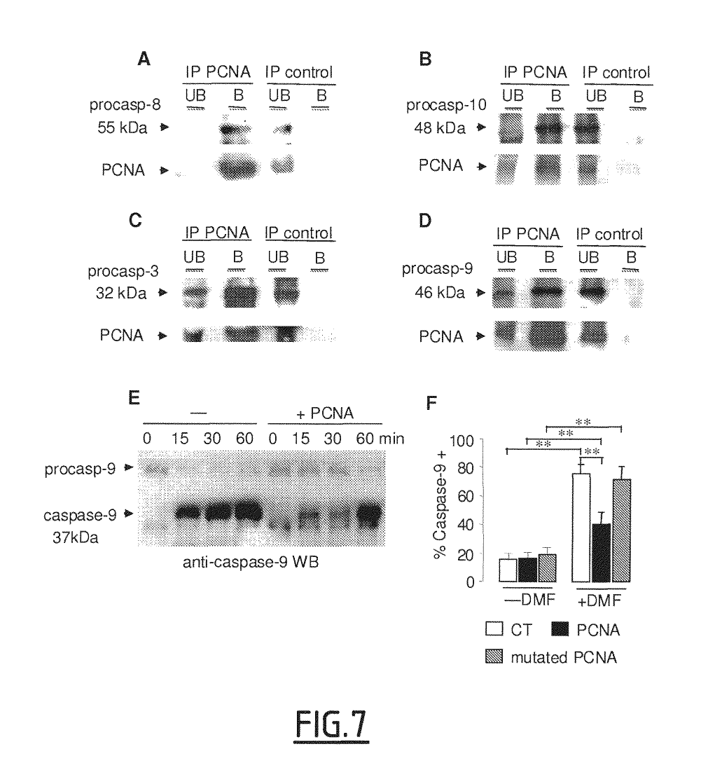

In contrast to this, using the same PCNA co-immunoprecipitation protocol, procaspase-8 (FIG. 7A), procaspase-10 (FIG. 7B), procaspase-3 (FIG. 7C) and procaspase-9 (FIG. 7D) could be detected using specific mAbs. In addition, the interaction was confirmed using the reciprocal immunoprecipitation experiment using an anti-procaspase IP and anti-PCNA for the Western blot analysis. Moreover, results from double immunolabeling with anti-PCNA and anti-procaspase performed on neutrophils strongly suggest that PCNA can colocalise with procaspase-8, -10, -3 and -9. Perhaps, the cytosolic PCNA-pro-caspase interaction might prevent their activation.

That hypothesis was tested by evaluating procaspase-9 cleavage in an in vitro assay, using neutrophil cytosol containing all the apoptosome components, i.e., procaspase-9 and apoptotic protease-activating factor-1 (Apaf-1) as previously described (McStay et al. 2008; Cell Death Differ. 15:322-331). Addition of exogenous purified cytochrome c and dATP triggered procaspase-9 cleavage (37 kDa) in a time-dependent manner, as evidenced by western blot analysis of caspase-9 (FIG. 7E). However, when purified PCNA was added to the mixture, procaspase-9 cleavage was delayed, with persistent procaspase-9 (47 kDa) and diminished 37-kDa fragment detection, thus demonstrating that purified PCNA impairs procaspase-9 activation. Ovalbumin tested at the same concentration (100 mM) did not affect pro-caspase 9 activation and served as control. Caspase-9 activation was next investigated in PL985 cells overexpressing PCNA or the interdomain connecting loop mutant. After 6 days of DMF exposure, caspase-9 activity was elevated unlike undifferentiated cells. This caspase-9 activity might correspond to physiological apoptosis of aged neutrophils. Notably, this differentiation-induced caspase-9 activation was significantly lower in PLB985-PCNA than in control PLB985, thus confirming the anti-apoptotic effect of PCNA. In contrast, no protective effect against differentiation-induced apoptosis was observed in PLB985 expressing the mutant PCNA (FIG. 7F).

Altogether, these findings strongly suggest that the interdomain connecting loop is involved in PCNA-procaspase-9 interaction.

### Example 9

### Peptides Derived from PCNA can Potentiate Neutrophil Apoptosis

The three-dimensional structure of PCNA was analyzed, and regions exposed at the surface of the protein were identified. Peptides derived from the sequence of these exposed regions of PCNA were synthesized. The sequences of these peptides are the followings:

(SEQ ID NO: 2)
APNQEKVSDYEMKLM
(SEQ ID NO: 3)
MKLMDLDVEQLGIPEQEYS
(SEQ ID NO: 4)
KDGVKF
(SEQ ID NO: 5)
SQTSNVDKEEEAV
(SEQ ID NO: 6)
MSADVPL
(SEQ ID NO: 7)
YYLAPKIEDEEGS

In order to facilitate entry of these peptides into the cells, a tag was fused to the peptide. Two different tags were used: either a Tat tag derived from the HIV-1 Tat polypeptide (YGRKKRRQRRR, SEQ ID NO: 27) was fused to the N-terminal of the peptide, or a RYIRS tag (SEQ ID NO: 8) was fused to the C-terminal of the peptide (see Table I).

TALBLE I

| | | SEQ | |
|---|---|---|---|
| Name | Sequence | ID No | Tag |
| peptide 1 | APNQEKVSDYEMKLMRYIRS | 9 | RYIRS |
| peptide 2 | YGRKKRRQRRRAPNQEKVSDYEMKLM | 10 | Tat |
| peptide 3 | MKLMDLDVEQLGIPEQEYSRYIRS | 11 | RYIRS |
| peptide 4 | YGRKKRRQRRRMKLMDLDVEQLGIPEQEYS | 12 | Tat |
| peptide 5 | KDGVKFRYIRS | 13 | RYIRS |
| peptide 6 | SQTSNVDKEEEAVRYIRS | 14 | RYIRS |
| peptide 7 | MSADVPLRYIRS | 15 | RYIRS |
| peptide 8 | YYLAPKIEDEEGSRYIRS | 16 | RYIRS |
| peptide 9 | YGRKKRRQRRRYYLAPKIEDEEGS | 17 | Tat |

The peptides were added to the neutrophil cells and their effect on cell apoptosis was analyzed by measuring the phosphatidylserine externalization. Phosphatidylserine was stained using a fluorochrome-coupled annexin V allowing detection of apoptotic neutrophils by flow cytometry. Neutrophils stained with annexin V express the phosphatidylserine and are recognized and phagocytosed by macrophages. Furthermore, a secondary staining with the 7AAD intercalating agent was used to indicate late apoptosis, allowing distinction between early and late apoptosis.

The results are shown in Table II to VI below.

TABLE II
Effect of the peptides on Total Apoptosis
(peptide concentration = 200 μM)

| Total | | Standard | | Student |
|---|---|---|---|---|
| Apoptosis | Mean | deviation | SEM | test |
| 4° C. | 7.84 | 2.63 | 1.31 | 8.28E−05 |
| overnight | 74.33 | 3.27 | 1.64 | |
| 37° C. | | | | |
| peptide 1 | 73.67 | 3.14 | 1.81 | 1.12E+00 |
| peptide 2 | 69.33 | 3.40 | 1.96 | 3.80E−01 |
| peptide 3 | 76.20 | 1.97 | 1.14 | 2.76E−01 |
| peptide 4 | 89.83 | 8.46 | 4.88 | 3.69E−02 |
| peptide 5 | 74.36 | 3.07 | 1.77 | 4.95E−01 |
| peptide 6 | 76.02 | 4.38 | 2.53 | 3.04E−01 |
| peptide 7 | 74.40 | 2.60 | 1.50 | 4.89E−01 |
| peptide 8 | 46.43 | 5.35 | 3.09 | 1.78E−03 |
| peptide 9 | 73.43 | 1.36 | 0.78 | 3.23E−01 |

TABLE III
Effect of the peptides on Early Apoptosis
(peptide concentration = 200 μM)

| Early | | Standard | | Student |
|---|---|---|---|---|
| Apoptosis | Mean | deviation | SEM | test |
| 4° C. | 7.96 | 3.47 | 1.73 | 4.55E−05 |
| overnight | 66.27 | 7.37 | 3.69 | |
| 37° C. | | | | |
| peptide 1 | 62.52 | 7.60 | 4.39 | 2.73E−01 |
| peptide 2 | 52.29 | 6.47 | 3.73 | 2.34E−02 |
| peptide 3 | 63.00 | 8.95 | 5.17 | 3.17E−01 |
| peptide 4 | 62.34 | 5.25 | 3.03 | 2.24E−01 |
| peptide 5 | 62.44 | 9.91 | 5.72 | 3.03E−01 |
| peptide 6 | 62.91 | 10.33 | 5.97 | 3.30E−01 |
| peptide 7 | 62.60 | 10.54 | 6.08 | 3.19E−01 |
| peptide 8 | 40.67 | 4.66 | 2.69 | 1.29E−03 |
| peptide 9 | 50.03 | 7.91 | 4.57 | 2.36E−02 |

TABLE IV
Effect of the peptides on Late Apoptosis
(peptide concentration = 200 μM)

| Late | | Standard | | Student |
|---|---|---|---|---|
| Apoptosis | Mean | deviation | SEM | test |
| 4° C. | 1.74 | 0.88 | 0.44 | 2.79E−02 |
| overnight | 9.02 | 4.87 | 2.44 | |
| 37° C. | | | | |
| peptide 1 | 11.47 | 7.33 | 4.23 | 3.24E−01 |
| peptide 2 | 17.38 | 3.18 | 1.84 | 2.06E−02 |
| peptide 3 | 13.27 | 8.46 | 4.88 | 2.47E−01 |
| peptide 4 | 27.88 | 4.44 | 2.56 | 1.87E−03 |
| peptide 5 | 12.36 | 9.11 | 5.26 | 3.03E−01 |
| peptide 6 | 13.11 | 9.68 | 5.59 | 2.77E−01 |
| peptide 7 | 12.30 | 9.66 | 5.58 | 3.15E−01 |
| peptide 8 | 6.23 | 4.31 | 2.49 | 2.31E−01 |
| peptide 9 | 23.90 | 7.84 | 4.53 | 4.34E−02 |

TABLE V
Effect of peptides 1-4 on Apoptosis
(peptide concentration = 500 μM)

| | Total | Early | Late |
|---|---|---|---|
| | Apoptosis | Apoptosis | Apoptosis |
| 4° C. | 10.9 | 10.02 | 2.97 |
| overnight | 71.74 | 68.06 | 6.16 |
| 37° C. | | | |
| peptide 1 | 68.74 | 65.06 | 6.62 |
| peptide 2 | 68.08 | 46.92 | 23.97 |
| peptide 3 | 78.56 | 68.05 | 12.63 |
| peptide 4 | 82.84 | 53.11 | 33.74 |

TABLE VI
Effect of peptides 5-9 on Apoptosis
(peptide concentration = 500 μM)

| | Total | Early | Late |
|---|---|---|---|
| | Apoptosis | Apoptosis | Apoptosis |
| 4° C. | 6.44 | 11.72 | 1.1 |
| overnight | 78.13 | 75.85 | 4.55 |
| 37° C. | | | |
| peptide 5 | 77.67 | 75.21 | 5.39 |
| peptide 6 | 67.42 | 66.43 | 5.2 |
| peptide 7 | 64.85 | 66.26 | 2.85 |
| peptide 8 | 48.77 | 48.94 | 8.99 |
| peptide 9 | 71.84 | 48.44 | 26.32 |

When added at a concentration of 200 μM, the peptide of sequence SEQ ID No. 12 (peptide 4) induced an increase of the annexin V staining and appeared to potentiate neutrophil apoptosis (Table II). Interestingly, this peptide corresponds to the p21 interacting site of PCNA. The pro-apoptotic activity of this peptide was particularly obvious on late apoptosis (Table IV).

Moreover, the peptide of sequence SEQ ID No. 11 (peptide 3), which corresponds to the same portion of the PCNA protein but bears a different tag, also induced an increase of the annexin V staining, when added at a concentration of 500 μM (Table V).

It has thus been demonstrated that peptides 3 and 4 exhibit a pro-apoptotic activity on neutrophils, and that they can thus be used for the treatment of inflammatory diseases.

In addition, it was found that peptide 8 exhibits a significant anti-apoptotic activity on neutrophils. This peptide, located at the PCNA carboxy terminus and triggering neutrophil survival, might act as a PCNA mimetic, stabilizing an anti-apoptotic protein. This activity might be useful in the case of an excess of neutrophil apoptosis as observed in neutropenia (cyclic or congenital neutropenia). Of note, these neutropenia are treated by G-CSF, a compound which stabilizes the PCNA scaffold in neutrophils (as shown in FIG. 3/7). Except G-CSF treatment, there is not other treatment for these neutropenia and no potential molecular mechanisms to explore to decrease the high level of neutrophil apoptosis. Thus, decreasing neutrophil apoptosis by interfering with specific PCNA partners whose binding sites are at the PCNA carboxy-terminus constitutes a promising therapeutical strategy.

### Example 10

### Discussion of the Results

Apoptosis regulation of the neutrophil lifespan provides a fine balance between their function as host defense effector cells and a safe turnover of these potentially harmful cells. Neutrophil apoptosis is essential for the resolution of inflammation and delayed apoptosis and compromised neutrophil clearance are, in fact, hallmarks of chronic inflammation. Therefore, the neutrophil has a carefully orchestrated lifespan, which requires the presence of a pre-established intracellular “clock” with successive steps and key control points, which are still undefined.

Herein, the inventors have found that PCNA, a well-known nuclear protein involved in DNA replication and repair, and considered as one of the central molecules responsible for life-or-death decision only in proliferating cells, displays all the features of a master regulator of neutrophil survival. Indeed, they observed that while circulating mature neutrophils contained elevated amounts of PCNA, their PCNA contents changed as a function of their viability status. In neutrophils undergoing apoptosis, PCNA was subjected to proteasome-mediated degradation, regardless of whether the trigger signalling the cascade passed through the extrinsic (death receptors) or the intrinsic pathway (mitochondria). By contrast, PCNA levels are stably maintained by factors that prolong neutrophil survival, e.g. G-CSF or IFN-g. Furthermore, in a murine model of LPS-induced pulmonary inflammation, PCNA levels in pre-apoptotic neutrophils isolated from BALF during inflammation resolution were much lower than those in fully viable neutrophils, purified from the bone marrow, supporting the idea that neutrophil PCNA levels vary during inflammation according to their survival status. Notably, modulation of PCNA contents, at least in human neutrophils, appears to involve only post-transcriptional events, since variations of PCNA mRNA expression were not detected during cytokine-induced neutrophil survival or physiological apoptosis. Similarly, PCNA gene induction has not been reported to occur in neutrophils isolated from donors treated with G-CSF in vivo. Herein, neutrophils isolated from G-CSF-treated donors exhibited both prolonged survival, but also high PCNA contents, in keeping with its major role in neutrophil survival. Whether or not PCNA is dispensable for neutrophil survival cannot be explored at present because of the lack of PCNA-knock out animals.

According to numerous studies, PCNA functions in the nucleus as a remarkable adaptor molecule that binds to numerous different proteins. Indeed, several authors established a central role for trimeric PCNA, a moving platform along the DNA molecule, as a communication center for a variety of cell nuclear processes, like DNA replication, nucleotide excision repair, post-replication mismatch repair and apoptosis.

In non-cycling cells, like neutrophils, PCNA anti-apoptotic activity should proceed via mechanisms theoretically not involving any nuclear function. And indeed, one salient feature uncovered in this study, was the exclusive localization of PCNA in the neutrophil cytosol, and the inventors documented its nucleus-to-cytosol relocalization at the later stages of neutrophil maturation. The underlying mechanisms either in terms of nucleo-cytoplasmic transport or sequestration mechanisms to retain PCNA within the cytoplasm compartment, remain to be deciphered. The inventors clearly demonstrated that, in primary neutrophils, PCNA was constitutively associated with procaspase-8, procaspase-10, procaspase-9, procaspase-3, presumably sequestering them within the cytosol to prevent their activation. Procaspase-9 sequestration by PCNA proves to be a very efficient way to inhibit apoptosis. However, one cannot exclude that association with other cytoplasmic proteins might mediate the PCNA anti-apoptotic effect in neutrophils.

In proliferating cells, PCNA is tightly controlled by the tumor suppressor protein p21, which was initially identified as a potent CDK inhibitor. Structural studies indicated that p21 directly blocks the surface region required for polymerase binding. Biochemical studies broadened that concept by showing that p21 is an effective competitor for other PCNA partners. Indeed, synthetic peptides carrying the consensus sequence for binding to the PCNA interdomain connecting loop, like the carboxyp21 peptide (residues 120-132 on PCNA sequence), stop progression through the cell cycle and induce apoptosis when transfected into proliferating cells. Herein, the inventors showed that carboxyp21 directly triggers neutrophil apoptosis and concomitant PCNA degradation; it also impaired the capacity of G-CSF to prolong neutrophil survival, thus clearly proving that PCNA is absolutely essential for the G-CSF anti-apoptotic effect in neutrophils.

These observations are also consistent with the report that roscovitine, a synthetic CDK inhibitor, triggers neutrophil apoptosis and, consequentially, promotes inflammation resolution in vivo. Hence, these authors have provided evidence that CDK activities, so far restricted to cell-cycle regulation, can control apoptosis in non proliferating cells, like neutrophils. Roscovitine has also been shown to improve the resolution of the inflammation after pneumococcal infection and accelerated recovery. Furthermore, although several cell-cycle proteins are down-regulated during neutrophil differentiation, mature neutrophils expressed high levels of CDK2 and the CDK inhibitor p27. Indeed, during inflammation, neutrophils are able to up-regulate survivin, a member of the inhibitor of apoptosis protein (IAP) family, which acts as a link between apoptosis and control of mitogenic progression in proliferating cells. Thus, cell-cycle regulatory proteins other than PCNA might be implicated in controlling neutrophil lifespan as well.

Most studies on neutrophils, viewed as potential cellular targets in inflammation, attempted to block their migration and influx. However, another aspect of neutrophil biology that could be targeted, albeit often underestimated, is the modulation of neutrophil survival. In this latter regard, an in-depth understanding of the specific and tightly regulated molecular mechanisms controlling neutrophil survival/death is required to intervene efficiently and effectively. The results reported herein are notable, not only for the demonstration that PCNA acts as a cytoplasmic platform pulling the strings of neutrophil survival but also because they expose to the light of day new ways to think about in neutrophils and their intracellular pathways in the context of inflammation or neutropenia.

### Example 11

### CDK2 or GADD45 siRNA Sensitized DMF-Differentiated PLB985 to Apoptosis

CDK2 and GADD45 have been described to be nuclear partners of PCNA in proliferating cells. The inventors have shown that both CDK2 and GADD45 were present within neutrophil cytosol and that decreasing the levels of CDK2 and GADD45 proteins using the siRNA technology sensitized neutrophil-differentiated PLB985 cells enhanced neutrophil-differentiated PLB985 cells to apoptosis.

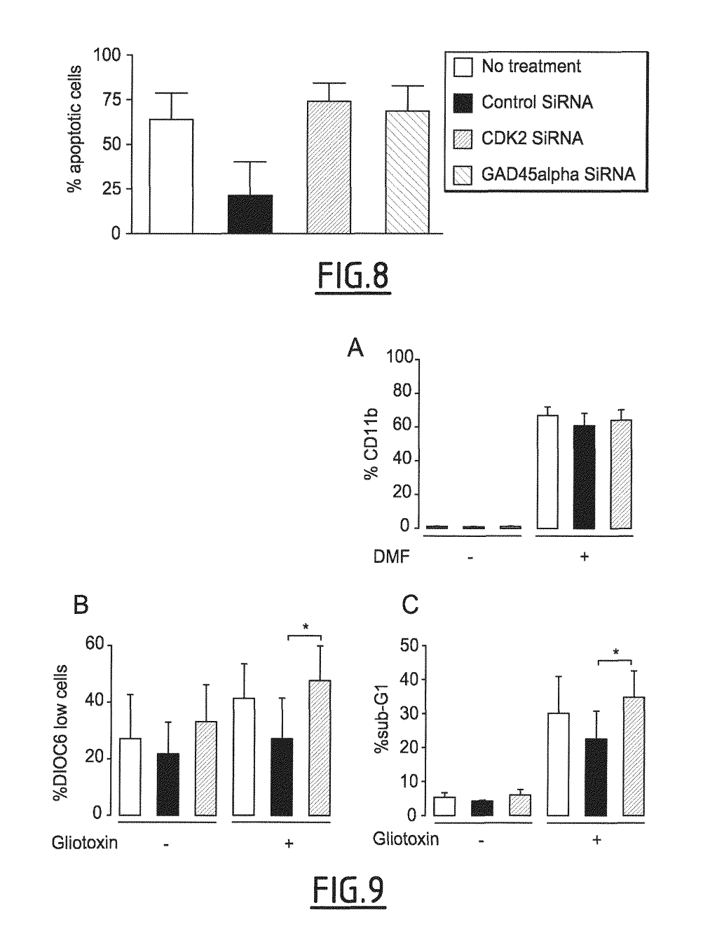

Notably, the siRNA procedure was performed on days 3 and 4 after DMF treatment, prior to analysis of the apoptosis rate on day 5. At this time point, all PCNA was cytoplasmic in DMF-differentiated PLB985 cells. No effect of CDK2 or GADD45 siRNA was observed on CD11b expression thus suggesting that it did not affect the differentiation process. CDK2 or GADD45 siRNA, but not scrambled siRNA, slightly but significantly enhanced the constitutive apoptosis that PLB985 cells undergo after differentiation, as assessed by mitochondria depolarization (FIG. 8).

These data confirm the new finding that interfering with PCNA partners, namely CDK2 and GADD45 alpha, can trigger neutrophil apoptosis.

### Example 12

### The Nuclear-to-Cytoplasmic Relocalization During Granulocyte Differentiation is Essential for PCNA Anti-Apoptotic Activity in Mature Neutrophils

The dimethylformamide (DMF)-differentiated PLB985 cells considered as “neutrophil-like cells” were used to study the impact of a modulation of PCNA export on PCNA anti-apoptotic activity. In order to investigate whether nuclear sequestration of PCNA would affect its PCNA anti-apoptotic activity in mature neutrophils, PLB985 cells were stably transfected with a nuclear PCNA mutant as referred to as SV40-NLS-PCNA; indeed, as previously described, the fusion of PCNA with the SV40 NLS results in its exclusive nuclear localization. Hence, we studied whether overexpression of SV40-NLS-PCNA would have an anti-apoptotic affect in differentiated PLB985, as previously described for wild type PCNA. As expected, overexpression of the SV40-NLS-PCNA mutant displaying an increased ability to enter the nucleus resulted in an increased PCNA nuclear localization in undifferentiated PLB985 cells as evidenced by direct fluorescence after PCNA immunolabelling. Likewise, an impaired nuclear-to-cytoplasmic relocalization was observed in DMF-differentiated PLB985-SV40NLS-PCNA as evidenced by an increased nuclear fluorescence as compared with PLB985-PCNA. The quantification of the nuclear immunofluorescence confirmed the significant increased observed in PLB985-SV40NLS-PCNA as compared with the PLB985-PCNA. However, despite this defect in the PCNA nuclear-to-cytoplasmic relocalization, no significant difference in the CD11b expression after DMF treatment was observed between PLB985-SV40NLS-PCNA and PLB985-PCNA. These data suggest that PCNA nuclear retention did not inhibit granulocytic differentiation as evaluated by CD11b expression (FIG. 9A). The inventors next studied whether this nuclear SV40NLS-PCNA mutant could still mediate its anti-apoptotic activity in neutrophil-differentiated PLB985. Apoptosis was triggered via the mitochondrial pathway using gliotoxin, a fungal toxin that directly binds to Bak to trigger mitochondria depolarization. The inventors confirmed that PLB985 cells overexpressing wild type PCNA were protected against gliotoxin-induced apoptosis, compared to PLB985 transfected with the control plasmid, as judged by mitochondria depolarization measured after Dioc6 labelling (FIG. 9B) and DNA fragmentation (FIG. 9C). As predicted, PLB985 expressing the nuclear SV40-NLS-PCNA mutant failed to display anti-apoptotic activity, thus corroborating the essential role of PCNA cytoplasmic subcellular localization for its anti-apoptotic activity in mature neutrophils.

1-10. (canceled)

11. A peptide consisting in an amino acid sequence selected from the group consisting of:
a) an amino acid sequence consisting of SEQ ID NO: 3 or 7;
b) an amino acid sequence consisting of SEQ ID NO: 3 or 7 fused to a tag;
c) an amino acid sequence consisting of SEQ ID NO: 11, SEQ ID NO: 12, SEQ ID NO: 16 or SEQ ID NO: 17;
d) a sequence homologous to the sequence of any one of (a) to (c) and presenting at least 80% of identity with said sequence of any one of (a) to (c); and
e) an amino acid sequence consisting of a fragment of at least 6 consecutive amino acids of the sequence of any one of (a) to (c).

12. A pharmaceutical composition comprising a peptide as defined in claim 11, and one or more physiologically acceptable carriers.

13. A method for the identification of a compound for use in the treatment of neutropenia or a disease involving an inflammatory process, comprising:
a) contacting a candidate compound with PCNA and at least one polypeptide liable to bind to PCNA;
b) comparing the quantity of PCNA bound to said at least one polypeptide liable to bind to PCNA in the presence and in the absence of the candidate compound; and
c) selecting the candidate compound if the quantity of PCNA bound to said at least one polypeptide liable to bind to PCNA is lower in the presence of said candidate compound than in the absence of said candidate compound.

14. The method according to claim 11, wherein the protein liable to bind to PCNA is selected from the group of p21, a peptide of SEQ ID NO: 23, a peptide of SEQ ID NO: 11, a peptide of SEQ ID NO: 12 and a peptide of SEQ ID NO: 16.

15. In vitro use of PCNA as a target for screening for drugs for the treatment of a disease involving an inflammatory process or neutropenia.

16. A method of treating a disease involving an inflammatory process in a subject in need thereof comprising
administering to said subject a therapeutically effective amount of a compound that inhibits interaction between Proliferating Cell Nuclear Antigen (PCNA) and at least one polypeptide liable to bind to PCNA.

17. The method of claim 16, wherein the compound inhibits an interaction between PCNA and at least one polypeptide liable to bind to PCNA at the interdomain connecting loop of PCNA.

18. The method of claim 16, wherein the compound is a peptide.

19. The method of claim 18, wherein the peptide is or comprises:
a) an amino acid sequence consisting of SEQ ID NO: 3 or 23;
b) an amino acid sequence consisting of SEQ ID NO: 3 or 23 fused to a tag;
c) an amino acid sequence consisting of SEQ ID NO: 11, SEQ ID NO: 12 or SEQ ID NO: 24;
d) a sequence homologous to the sequence of any one of (a) to (c) and presenting at least 80% of identity with said sequence of any one of (a) to (c); and
e) an amino acid sequence consisting of a fragment of at least 6 consecutive amino acids of the sequence of any one of (a) to (c).

20. The method of claim 16, wherein the disease involving an inflammatory process is mediated by neutrophil cells.

21. The method of claim 16, wherein the disease involving an inflammatory process is selected from the group consisting of cardiac ischemia, bacterial endocarditis, purulent pericarditis, polyarteritis nodosa, Kawasaki disease, leucytoclastic vasculitis, microscopic polyangiitis, bacillary angiomatosis, adult respiratory distress syndrome, cystic fibrosis, bronchopneumonia, ulcer, Crohn's disease, burns, acute gout, pseudogout, infectious arthritis, seronegative spondyloarthritides, rheumatoid polyarthritis, juvenile arthritis, osteoarthritis and transplant rejection.

22. The method of claim 16, wherein the compound induces apoptosis of neutrophil cells.

23. A method of treating neutropenia in a subject in need thereof, comprising
administering to said subject a therapeutically effective amount of a peptide comprising the carboxy-terminal region of PCNA or a fragment of at least six consecutive amino acids thereof, said carboxy-terminal region of PCNA consisting of amino acids 249 to 261 of SEQ ID NO: 1.

24. The method of claim 23, wherein the peptide comprises:
a) an amino acid sequence consisting of SEQ ID NO: 7;
b) an amino acid sequence consisting of SEQ ID NO: 7 fused to a tag;
c) an amino acid sequence consisting of SEQ ID NO: 16 or SEQ ID NO: 17;
d) a sequence homologous to the sequence of any one of (a) to (c) and presenting at least 80% of identity with said sequence of any one of (a) to (c); and
e) an amino acid sequence consisting of a fragment of at least 6 consecutive amino acids of the sequence of any one of (a) to (c).

25. The method of claim 23, wherein the peptide prevents apoptosis of neutrophil cells.


SEQUENCE LISTING (29 sequences)

SEQ ID NO: 1 - protein, 261 aa, Homo sapiens
  MISC_FEATURE (105)..(110): TRAF2 binding (cytosol)
MFEARLVQGSILKKVLEALKDLINEACWDISSSGVNLQSMDSSHVSLVQLTLRSEGFDTYRCDRNLAMGVNLTSMSKILKCAGNEDIITLRAEDNADTLALVFEAPNQEKVSDYEMKLMDLDVEQLGIPEQEYSCVVKMPSGEFARICRDLSHIGDAVVISCAKDGVKFSASGELGNGNIKLSQTSNVDKEEEAVTIEMNEPVQLTFALRYLNFFTKATPLSSTVTLSMSADVPLVVEYKIADMGHLKYYLAPKIEDEEGS

SEQ ID NO: 2 - protein, 15 aa, Artificial
  Synthetic peptide
APNQEKVSDYEMKLM

SEQ ID NO: 3 - protein, 19 aa, Artificial
  Synthetic peptide
MKLMDLDVEQLGIPEQEYS

SEQ ID NO: 4 - protein, 6 aa, Artificial
  Synthetic peptide
KDGVKF

SEQ ID NO: 5 - protein, 13 aa, Artificial
  Synthetic peptide
SQTSNVDKEEEAV

SEQ ID NO: 6 - protein, 7 aa, Artificial
  Synthetic peptide
MSADVPL

SEQ ID NO: 7 - protein, 13 aa, Artificial
  Synthetic peptide
YYLAPKIEDEEGS

SEQ ID NO: 8 - protein, 5 aa, Artificial
  Synthetic peptide tag
RYIRS

SEQ ID NO: 9 - protein, 20 aa, Artificial
  Synthetic peptide
APNQEKVSDYEMKLMRYIRS

SEQ ID NO: 10 - protein, 26 aa, Artificial
  Synthetic peptide
YGRKKRRQRRRAPNQEKVSDYEMKLM

SEQ ID NO: 11 - protein, 24 aa, Artificial
  Synthetic peptide
MKLMDLDVEQLGIPEQEYSRYIRS

SEQ ID NO: 12 - protein, 30 aa, Artificial
  Synthetic peptide
YGRKKRRQRRRMKLMDLDVEQLGIPEQEYS

SEQ ID NO: 13 - protein, 11 aa, Artificial
  Synthetic peptide
KDGVKFRYIRS

SEQ ID NO: 14 - protein, 18 aa, Artificial
  Synthetic peptide
SQTSNVDKEEEAVRYIRS

SEQ ID NO: 15 - protein, 12 aa, Artificial
  Synthetic peptide
MSADVPLRYIRS

SEQ ID NO: 16 - protein, 18 aa, Artificial
  Synthetic peptide
YYLAPKIEDEEGSRYIRS

SEQ ID NO: 17 - protein, 24 aa, Artificial
  Synthetic peptide
YGRKKRRQRRRYYLAPKIEDEEGS

SEQ ID NO: 18 - protein, 13 aa, Artificial
  Synthetic peptide
RQTSMTDFYHSKR

SEQ ID NO: 19 - protein, 13 aa, Artificial
  Synthetic peptide
RQTGETDFDHAKA

SEQ ID NO: 20 - protein, 16 aa, Artificial
  Synthetic peptide
SAVLQKKITDYFHPKK

SEQ ID NO: 21 - protein, 13 aa, Artificial
  Synthetic peptide
RQTSMTDFYHSKR

SEQ ID NO: 22 - protein, 13 aa, Artificial
  Synthetic peptide
RQTGETDFDHAKA

SEQ ID NO: 23 - protein, 20 aa, Artificial
  Synthetic peptide
KRRQTSMTDFYHSKRRLIFS

SEQ ID NO: 24 - protein, 25 aa, Artificial
  Synthetic peptide
KRRQTSMTDFYHSKRRLIFSRYIRS

SEQ ID NO: 25 - protein, 25 aa, Artificial
  Synthetic peptide
KRRQTGETDFDHAKAALIFSRYIRS

SEQ ID NO: 26 - protein, 164 aa, Homo sapiens
MSEPAGDVRQNPCGSKACRRLFGPVDSEQLSRDCDALMAGCIQEARERWNFDFVTETPLEGDFAWERVRGLGLPKLYLPTGPRRGRDELGGGRRPGTSPALLQGTAEEDHVDLSLSCTLVPRSGEQAEGSPGGPGDSQGRKRRQTSMTDFYHSKRRLIFSKRKP

SEQ ID NO: 27 - protein, 11 aa, Artificial
  Synthetic peptide tag
YGRKKRRQRRR

SEQ ID NO: 28 - DNA, 88 bp, Artificial
  Synthetic oligonucleotide
ggccgcgccatgccgaagaagaagcgcaaagtaggcgaagggcaagggcaagggcaagggccgggccgcggctacgcgtatcgatccc

SEQ ID NO: 29 - DNA, 88 bp, Artificial
  Synthetic oligonucleotide
tcgagggatcgatacgcgtagccgcggcccggcccttgcccttgcccttgcccttcgcctactttgcgcttcttcttcggcatggcgc